(19) United States
(12) Patent Application Publication
(10) Pub. No.: US 2025/0345389 A1
(43) Pub. Date: Nov. 13, 2025

(54) COMBINATION THERAPIES

(71) Applicant: Gilead Sciences, Inc., Foster City, CA (US)

(72) Inventors: Rudolf K.F. Beran, Foster City, CA (US); Simon P. Fletcher, San Mateo, CA (US); Meghan M. Holdorf, Oakland, CA (US); Robert C. Muench, Hayward, CA (US)

(21) Appl. No.: 19/203,264

(22) Filed: May 9, 2025

Related U.S. Application Data
(60) Provisional application No. 63/646,265, filed on May 13, 2024.

(51) Int. Cl. A61K 38/16; A61P 31/14; C12N 15/113

(52) U.S. Cl. CPC A61K 38/16; A61P 31/14; C12N 15/1131

(57) ABSTRACT

The present application provides combinations of bulevirtide, or a pharmaceutically acceptable salt thereof, and an inhibitory nucleic acid targeting hepatitis B virus (HBV), useful for treating hepatitis D virus (HDV) infection.

### SEQUENCE LISTING

This application contains a Sequence Listing that has been submitted electronically as an XML file named 35648-0305001_SL_ST26.xml. The XML file, created on May 5, 2025, is 13,923 bytes in size. The material in the XML file is hereby incorporated by reference in its entirety.

### TECHNICAL FIELD

The present application provides combinations of bulevirtide, or a pharmaceutically acceptable salt thereof, and an inhibitory nucleic acid targeting hepatitis B virus (HBV), which are useful for the inhibition of hepatitis B virus (HBV) and/or hepatitis D virus (HDV) infection, prevention of primary HBV and/or HDV infection, as well as treatment of (chronic) hepatitis B and/or D.

### BACKGROUND

Hepatitis B virus (HBV) is the prototype of a family of small, enveloped DNA viruses of mammals and birds (Seeger et al, Microbiol. Mol. Biol. Rev. 64, 51-68 (2000)). Hepatitis D virus (HDV) is a negative sense single stranded circular RNA satellite virus of HBV that is encapsulated by envelope proteins encoded by HBV. HDV depends on the HBV envelope for assembly, infection, and extracellular viral spread. The HBV envelope consists of three proteins termed L-(large), M-(middle) and S-(small) surface antigen (HBsAg) derived from the same open reading frame with a common C terminal domain. The M- and L-protein carry additional N-terminal extensions of 55 (preS2) and, genotype-dependent, 107 or 118 aa (preS1). During synthesis, the preS1 domain of L is myristoylated and translocated to the ER membrane. This modification is essential for HBV and HDV viral entry through the host sodium taurocholate cotransporting polypeptide (NTCP) on hepatocytes (Gripon et al, Virology, 213, 292-299; and Le Seyec et al, J. Virol. 73, 2052-2057 (1999)). Besides virions, smaller non-infectious subviral particles (SVPs) mainly composed of S-HBsAg are also present in the serum of HBV and HDV-infected patients in large abundance.

### SUMMARY

The present application provides, inter alia, a method of treating hepatitis D virus (HDV) infection in a patient, comprising administering to the patient:
i) bulevirtide, or a pharmaceutically acceptable salt thereof; and
ii) an inhibitory nucleic acid targeting hepatitis B virus (HBV).

The present application further provides one or more pharmaceutical compositions comprising bulevirtide, or a pharmaceutically acceptable salt thereof; and an inhibitory nucleic acid targeting hepatitis B virus (HBV), and one or more pharmaceutically acceptable excipients.

The present application further provides a combination of bulevirtide, or a pharmaceutically acceptable salt thereof; and an inhibitory nucleic acid targeting hepatitis B virus (HBV), for use in any of the methods described herein.

The present application further provides a combination of bulevirtide, or a pharmaceutically acceptable salt thereof; and an inhibitory nucleic acid targeting hepatitis B virus (HBV), for the preparation of one or more medicaments for use in any of the methods described herein.

### DESCRIPTION OF DRAWINGS

FIG. 1 shows the structure of bulevirtide (BLV).

FIG. 2 shows HDV antiviral activity in an HBV/HDV co-infected PHH spread assay with an HBV siRNA.

FIG. 3 shows HDV antiviral activity of HBV siRNA alone and in combination with BLV in an HBV/HDV co-infected PHH spread assay.

FIG. 4 shows HDV antiviral activity in vivo with BLV and HBV siRNA alone and in combination.

### DETAILED DESCRIPTION

The description below is made with the understanding that the present disclosure is to be considered as an exemplification of the claimed subject matter and is not intended to limit the appended claims to the specific embodiments illustrated. The headings used throughout this disclosure are provided for convenience and are not to be construed to limit the claims in any way. Embodiments illustrated under any heading may be combined with embodiments illustrated under any other heading.

The present application provides a method of treating hepatitis D virus (HDV) infection in a patient, comprising administering to the patient:
i) bulevirtide, or a pharmaceutically acceptable salt thereof; and
ii) an inhibitory nucleic acid targeting hepatitis B virus (HBV) (e.g., an inhibitory nucleic acid targeting HBc, HBx, HBsAg, or a combination of any of these).

Bulevirtide is a hydrophobic modified preS-derived peptide of HBV having the structure of Formula I:

Myr-Gly-Thr-Asn-Leu-Ser-Val-Pro-Asn-Pro-Leu-Gly-Phe-Phe-Pro-Asp-His-Gln-Leu-Asp-Pro-Ala-Phe-Gly-Ala-Asn-Ser-Asn-Asn-Pro-Asp-Trp-Asp-Phe-Asn-Pro-Asn-Lys-Asp-His-Trp-Pro-Glu-Ala-Asn-Lys-Val-Gly-NH₂ I.

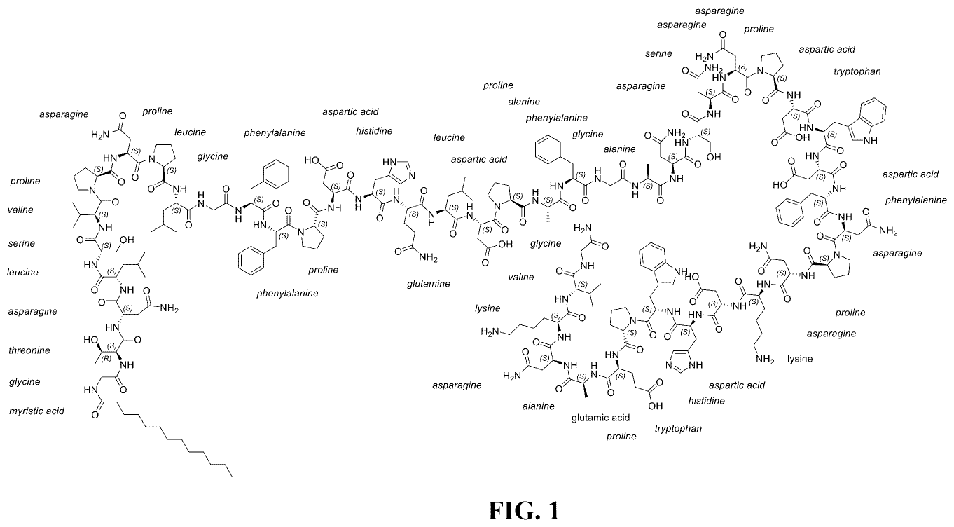

As used herein, “Myr” refers to a coupled myristic acid group (i.e., C₁₃H₂₇C(O)—). As used herein, bulevirtide may also be referred to as “the compound of Formula I”, “Myr-(SEQ ID NO:1)-NH₂”, or “C₁₃H₂₇C(O)-(SEQ ID NO:1)-NH₂”. The structure of bulevirtide is shown in FIG. 1.

As used herein, SEQ ID NO:1 refers to the following sequence: Gly-Thr-Asn-Leu-Ser-Val-Pro-Asn-Pro-Leu-Gly-Phe-Phe-Pro-Asp-His-Gln-Leu-Asp-Pro-Ala-Phe-Gly-Ala-Asn-Ser-Asn-Asn-Pro-Asp-Trp-Asp-Phe-Asn-Pro-Asn-Lys-Asp-His-Trp-Pro-Glu-Ala-Asn-Lys-Val-Gly (SEQ ID NO:1).

Additional methods of preparing bulevirtide can be found, for example, in U.S. Pat. No. 9,562,076, the disclosure of which is incorporated herein by reference in its entirety.

### Inhibitory Nucleic Acids Targeting HBV

### (i) Hepatitis B Core Protein (HBc) and Inhibitory Nucleic Acids Targeting

HBc plays an important role in the viral life cycle, particularly serving as the basic unit for capsid assembly, and HBc is closely associated with HBV genome replication and progeny virion production. An inhibitory nucleic acid targeting HBc can be used in methods for treating HDV infection in a patient.

An exemplary sequence of a HBc nucleic acid is provided in nucleic acids 1814 to 2452 of GenBank at Accession No. AF121249.1, which is provided below as SEQ ID NO:2.

(SEQ ID NO: 2)
ATGCAACTTTTTCACCTCTGCCTAATCATCTCATGTTCATGTCCTACTGTTCAAGCCTCCAAGCTGTGCCT
TGGGTGGCTTTGGGGCATGGACATTGACCCGTATAAAGAATTTGGAGCTTCTGTGGAGTTACTCTCTTTTT
TGCCTTCTGACTTCTTTCCTTCTATTCGAGATCTCCTCGACACCGCCTCTGCTCTGTATCGGGAGGCCTTA
GAGTCTCCGGAACATTGTTCACCTCACCATACGGCACTCAGGCAAGCTATTTTGTGTTGGGGTGAGTTAAT
GAATCTGGCCACCTGGGTGGGAAGTAATTTGGAAGATCCAGCATCCAGGGAATTAGTAGTCAGTTATGTCA
ACGTTAATATGGGCCTAAAAATCAGACAACTATTGTGGTTTCACATTTCCTGTCTTACTTTTGGGAGAGAA
ACTGTTCTTGAATATTTGGTGTCTTTTGGAGTGTGGATTCGCACTCCTCCTGCATATAGACCACCAAATGC
CCCTATCTTATCAACACTTCCGGAAACTACTGTTGTTAGACGAAGAGGCAGGTCCCCTAGAAGAAGAACTC
CCTCGCCTCGCAGACGAAGGTCTCAATCGCCGCGTCGCAGAAGATCTCAATCTCGGGAATCTCAATGTTAG.

An inhibitory nucleic acid targeting HBc for use in methods described herein can be single-stranded or double-stranded. The inhibitory nucleic acid targeting HBc can be any length suitable for inhibiting HBc expression. In some embodiments, the inhibitory nucleic acid targeting HBc comprises between 15 and 25 nucleic acids in length, e.g., 15, 16, 17, 18, 19, 20, 21, 22, 23, 24, or 25 nucleic acids in length.

In some embodiments, the inhibitory nucleic acid targeting HBc comprises a sense strand. In such instances, the inhibitory nucleic acid targeting HBc comprises at least 8 consecutive nucleic acids of the HBc nucleic acid sequence provided as SEQ ID NO:2, e.g., at least 9, 10, 11, 12, 14, 15, 16, 17, 18, 19, 20, 21, 22, 23, 24, or 25 consecutive nucleic acids of the HBc nucleic acid sequence provided as SEQ ID NO:2. For example, in some embodiments, the inhibitory nucleic acid targeting HBc comprises CCUCUGCCUAAUCAUCUC (SEQ ID NO:3).

In some embodiments, the inhibitory nucleic acid targeting HB comprises the RNA equivalent of at least 8 consecutive nucleic acids of the HBc nucleic acid sequence provided as SEQ ID NO:3.

In some embodiments, the inhibitory nucleic acid targeting HBc comprises an antisense strand. In such instances, the inhibitory nucleic acid targeting HBc comprises at least 8 consecutive nucleic acids that are complementary to the HBc nucleic acid sequence provided as SEQ ID NO:2.

Non-limiting examples of nucleic acid molecules for inhibiting expression of HBc that can be used in methods described herein include those disclosed in U.S. Pat. No. 11,492,623.

In some embodiments, an inhibitory nucleic acid targeting HBc used in the methods described herein inhibits HBc expression by at least 10% (e.g., at least 20%, at least 30%, at least 40%, at least 50%, at least 60%, at least 70%, at least 80%, at least 90%, or more). In some embodiments, an inhibitory nucleic acid targeting HBc used in the methods described herein inhibits HBV infection by at least 10% (e.g., at least 20%, at least 30%, at least 40%, at least 50%, at least 60%, at least 70%, at least 80%, at least 90%, or more).

In some embodiments, the inhibitory nucleic acid targeting HBc is a nucleic acid molecule such as a short interfering nucleic acid (siNA), a short interfering RNA (siRNA), a double-stranded RNA (dsRNA), a micro-RNA (miRNA), or a short hairpin RNA (shRNA) molecules that binds to HBc nucleic acid and inhibit expression of HBc. Such nucleic acid molecules can include non-naturally-occurring nucleobases (e.g., modified nucleobases), sugars (e.g., substituted sugar moieties), and/or covalent internucleoside linkages (e.g., modified backbones). In some embodiments, the inhibitory nucleic acid targeting HBc is chemically modified to enhance stability or other beneficial characteristics.

In some embodiments, the inhibitory nucleic acid targeting HBc further comprises a ligand. In some embodiments, the ligand is conjugated to the 3′ end of the sense strand of the inhibitory nucleic acid. In some embodiments, the ligand is an N-acetylgalactosamine (GalNAc) derivative.

### (ii) Hepatitis B X Protein (HBx) and Inhibitory Nucleic Acids Targeting HBx

Hepatitis B X protein (HBx) is a 154-amino acid regulatory protein of molecular weight 17 kDa. HBx plays an important role in the viral life cycle in that it targets the host DNA-binding complex Smc5/6 for proteosomal degradation, thus permitting HBV cccDNA transcription. Any inhibitory nucleic acid targeting HBx can be used in methods for treating HDV infection in a patient as it will target all forms of HBV mRNA including HBsAg, which is required for the viral envelope.

An exemplary sequence of a HBx nucleic acid is provided in nucleic acids 1374 to 1838 of GenBank at Accession No. AF121249.1, which is provided below as SEQ ID NO:5.

(SEQ ID NO: 5)
ATGGCTGCTAGGCTGTGCTGCCAACTGGATCCTGCGCGGGACGTCCTTTGTTTACGTCCCGTCGGCGCTGA
ATCCCGCGGACGACCCCTCCCGGGGCCGCTTGGGGCTCTACCGCCCGCTTCTCCGCCTATTGTACCGTCCG
ACCACGGGGCGCACCTCTCTTTACGCGGACTCCCCGTCTGTGCCTTCTCATCTGCCGGACCGTGTGCACTT
CGCTTCACCTCTGCACGTCGCATGGAGACCACCGTGAACGCCCACGGGAACCTGCCCAAGGTCTTGCATAA
GAGGACTCTTGGACTTTCAGCAATGTCAACGACCGACCTTGAGGCATACTTCAAAGACTGTGTGTTTACTG
AGTGGGAGGAGTTGGGGGAGGAGGTTAGGTTAAAGGTCTTTGTACTAGGAGGCTGTAGGCATAAATTGGTG
TGTTCACCAGCACCATGCAACTTTTTCACCTCTGCCTAA.

An inhibitory nucleic acid targeting HBx for use in methods described herein can be single-stranded or double-stranded. The inhibitory nucleic acid targeting HBx can be any length suitable for inhibiting HBx expression. In some embodiments, the inhibitory nucleic acid targeting HBx comprises between 15 and 25 nucleic acids in length, e.g., 15, 16, 17, 18, 19, 20, 21, 22, 23, 24, or 25 nucleic acids in length.

In some embodiments, the inhibitory nucleic acid targeting HBx comprises a sense strand. In such instances, the inhibitory nucleic acid targeting HBx comprises at least 8 consecutive nucleic acids of the HBx nucleic acid sequence provided as SEQ ID NO:5, e.g., at least 9, 10, 11, 12, 14, 15, 16, 17, 18, 19, 20, 21, 22, 23, 24, or 25 consecutive nucleic acids of the HBx nucleic acid sequence provided as SEQ ID NO:5. For example, in some embodiments, the inhibitory nucleic acid targeting HBx comprises CCUCUGCCUAAUCAUCUC (SEQ ID NO:3).

In some embodiments, the inhibitory nucleic acid targeting HBx comprises the RNA equivalent of at least 8 consecutive nucleic acids of the HBx nucleic acid sequence provided as SEQ ID NO:5. For example, in some embodiments, the inhibitory nucleic acid targeting HBc comprises ACCUCUGCCUAAUCAUCUC (SEQ ID NO:4).

In some embodiments, the inhibitory nucleic acid targeting HBx comprises an antisense strand. In such instances, the inhibitory nucleic acid targeting HBx comprises at least 8 consecutive nucleic acids that are complementary to the HBx nucleic acid sequence provided as SEQ ID NO:5.

Non-limiting examples of nucleic acid molecules for inhibiting expression of HBx that can be used in methods described herein include those disclosed in U.S. Pat. No. 11,492,623.

In some embodiments, an inhibitory nucleic acid targeting HBx used in the methods described herein inhibits HBx expression by at least 10% (e.g., at least 20%, at least 30%, at least 40%, at least 50%, at least 60%, at least 70%, at least 80%, at least 90%, or more). In some embodiments, an inhibitory nucleic acid targeting HBx used in the methods described herein inhibits HBV infection by at least 10% (e.g., at least 20%, at least 30%, at least 40%, at least 50%, at least 60%, at least 70%, at least 80%, at least 90%, or more).

In some embodiments, the inhibitory nucleic acid targeting HBx is a nucleic acid molecule such as a short interfering nucleic acid (siNA), a short interfering RNA (siRNA), a double-stranded RNA (dsRNA), a micro-RNA (miRNA), or a short hairpin RNA (shRNA) molecules that binds to HBx nucleic acid and inhibit expression of HBx. Such nucleic acid molecules can include non-naturally-occurring nucleobases (e.g., modified nucleobases), sugars (e.g., substituted sugar moieties), and/or covalent internucleoside linkages (e.g., modified backbones). In some embodiments, the inhibitory nucleic acid targeting HBx is chemically modified to enhance stability or other beneficial characteristics.

In some embodiments, the inhibitory nucleic acid targeting HBx further comprises a ligand. In some embodiments, the ligand is conjugated to the 3′ end of the sense strand of the inhibitory nucleic acid. In some embodiments, the ligand is an N-acetylgalactosamine (GalNAc) derivative.

In some embodiments, the inhibitory nucleic acid is complementary to at least 8 consecutive nucleic acids of a HBx mRNA sequence.

In some embodiments, the inhibitory nucleic acid comprises at least 15 consecutive nucleic acids from the nucleic acid sequence set forth in SEQ ID NO:5.

In some embodiments, inhibitory nucleic acid comprises between 15 and 25 nucleic acids in length.

In some embodiments, the inhibitory nucleic acid comprises an antisense oligonucleotide, a short interfering RNA (siRNA), or a short hairpin RNA (shRNA).

In some embodiments, the inhibitory nucleic acid is single-stranded or double-stranded.

In some embodiments, the inhibitory nucleic acid comprises one or more modifications.

(iii) Hepatitis B Surface Antigen (HBsAg) and Inhibitory Nucleic Acids Targeting HBsAg

HBsAg is a protein on the surface of the HBV that can be detected in high levels in serum during acute or chronic HBV infection. Any inhibitory nucleic acid targeting HBsAg can be used in methods for treating HDV infection in a patient.

An exemplary sequence of a HBsAg nucleic acid is provided in nucleic acids 2854 to 835 of GenBank at Accession No. X70185.1, which is provided below as SEQ ID NO:6.

(SEQ ID NO: 6)
ATGGGAGGTTGGTCATCAAAACCTCGCAAAGGCATGGGGACGAATCTTTCTGTTCCCAACCCTCTGGGATTCTTTCC
CGATCATCAGTTGGACCCTGCATTCGGAGCCAACTCAAACAATCCAGATTGGGACTTCAACCCCATCAAGGACCACT
GGCCAGCAGCCAACCAGGTAGGAGTGGGAGCATTCGGGCCAAGGCTCACCCCTCCACACGGCGGTATTTTGGGTGGA
GCCCTCAGGCTCAGGGCATATTGACCACAGTGTCAACAATTCCTCCTCCTGCCTCCACCAATCGGCAGTCAGGAAGG
CAGCCTACTCCCATCTCTCCACCTCTAAGAGACAGTCATCCTCAGGCCATGCAGTGGAATTCCACTGCCTTCCACCA
AGCTCTGCAGGATCCCAAAGTCAGGGGTCTGTATCTTCCTGCTGGTGGCTCCAGTTCAGGAACAGTAAACCCTGCTC
CGAATATTGCCTCTCACATCTCGTCAATCTCCGCGAGGACTGGGGACCCTGTGACGAACATGGAGAACATCACATCA
GGATTCCTAGGACCCCTGCTCGTGTTACAGGCGGGGTTTTTCTTGTTGACAAGAATCCTCACAATACCGCAGAGTCT
AGACTCGTGGTGGACTTCTCTCAATTTTCTAGGGGGGTCACCCGTGTGTCTTGGCCAAAATTCGCAGTCCCCAACCT
CCAATCACTCACCAACCTCCTGTCCTCCAATTTGTCCTGGTTATCGCTGGATGTGTCTGCGGCGTTTTATCATATTC
CTCTTCATCCTGCTGCTATGCCTCATCTTCTTGTTGGTTCTTCTGGATTATCAAGGTATGTTGCCCGTTTGTCCTCT
AATTCCAGGATCAACAACAACCAGTACGGGACCATGCAAAACCTGCACGACTCCTGCTCAAGGCAACTCTATGTTTC
CCTCATGTTGCTGTACAAAACCTACGGATGGAAATTGCACCTGTATTCCCATCCCATCGTCCTGGGCTTTCGCAAAA
TACCTATGGGAGTGGGCCTCAGTCCGTTTCTCTTGGCTCAGTTTACTAGTGCCATTTGTTCAGTGGTTCGTAGGGCT
TTCCCCCACTGTTTGGCTTTCAGCTATATGGATGATGTGGTATTGGGGGCCAAGACTGTACAGCATCGTGAGTCCCT
TTATACCGCTGTTACCAATTTTCTTTTGTCTCTGGGTATACATTTAA.

An inhibitory nucleic acid targeting HBsAg for use in methods described herein can be single-stranded or double-stranded. The inhibitory nucleic acid targeting HBsAg can be any length suitable for inhibiting HBsAg expression. In some embodiments, the inhibitory nucleic acid targeting HBsAg comprises between 15 and 25 nucleic acids in length, e.g., 15, 16, 17, 18, 19, 20, 21, 22, 23, 24, or 25 nucleic acids in length.

In some embodiments, the inhibitory nucleic acid targeting HBsAg comprises a sense strand. In such instances, the inhibitory nucleic acid targeting HBsAg comprises at least 8 consecutive nucleic acids of the HBsAg nucleic acid sequence provided as SEQ ID NO:6, e.g., at least 9, 10, 11, 12, 14, 15, 16, 17, 18, 19, 20, 21, 22, 23, 24, or 25 consecutive nucleic acids of the HBsAg nucleic acid sequence provided as SEQ ID NO:6. For example, in some embodiments, the inhibitory nucleic acid targeting HBsAg comprises CCUCUGCCUAAUCAUCUC (SEQ ID NO:3).

In some embodiments, the inhibitory nucleic acid targeting HBsAg comprises the RNA equivalent of at least 8 consecutive nucleic acids of the HBsAg nucleic acid sequence provided as SEQ ID NO:6.

In some embodiments, the inhibitory nucleic acid targeting HBsAg comprises an antisense strand. In such instances, the inhibitory nucleic acid targeting HBsAg comprises at least 8 consecutive nucleic acids that are complementary to the HBsAg nucleic acid sequence provided as SEQ ID NO:6.

Non-limiting examples of nucleic acid molecules for inhibiting expression of HBsAg that can be used in methods described herein include those disclosed in U.S. Pat. No. 11,492,623.

In some embodiments, an inhibitory nucleic acid targeting HBsAg used in the methods described herein inhibits HBsAg expression by at least 10% (e.g., at least 20%, at least 30%, at least 40%, at least 50%, at least 60%, at least 70%, at least 80%, at least 90%, or more). In some embodiments, an inhibitory nucleic acid targeting HBsAg used in the methods described herein inhibits HBV infection by at least 10% (e.g., at least 20%, at least 30%, at least 40%, at least 50%, at least 60%, at least 70%, at least 80%, at least 90%, or more).

In some embodiments, the inhibitory nucleic acid targeting HBsAg is a nucleic acid molecule such as a short interfering nucleic acid (siNA), a short interfering RNA (siRNA), a double-stranded RNA (dsRNA), a micro-RNA (miRNA), or a short hairpin RNA (shRNA) molecules that binds to HBsAg nucleic acid and inhibit expression of HBsAg. Such nucleic acid molecules can include non-naturally-occurring nucleobases (e.g., modified nucleobases), sugars (e.g., substituted sugar moieties), and/or covalent internucleoside linkages (e.g., modified backbones). In some embodiments, the inhibitory nucleic acid targeting HBsAg is chemically modified to enhance stability or other beneficial characteristics.

In some embodiments, the inhibitory nucleic acid targeting HBsAg further comprises a ligand. In some embodiments, the ligand is conjugated to the 3′ end of the sense strand of the inhibitory nucleic acid. In some embodiments, the ligand is an N-acetylgalactosamine (GalNAc) derivative.

In some embodiments, the inhibitory nucleic acid is complementary to at least 8 consecutive nucleic acids of a HBsAg mRNA sequence.

In some embodiments, the inhibitory nucleic acid comprises at least 15 consecutive nucleic acids from the nucleic acid sequence set forth in SEQ ID NO:6.

In some embodiments, inhibitory nucleic acid comprises between 15 and 25 nucleic acids in length.

In some embodiments, the inhibitory nucleic acid comprises an antisense oligonucleotide, a short interfering RNA (siRNA), or a short hairpin RNA (shRNA).

In some embodiments, the inhibitory nucleic acid is single-stranded or double-stranded.

In some embodiments, the inhibitory nucleic acid comprises one or more modifications.

In some embodiments, the inhibitory nucleic acid is complementary to at least 8 consecutive nucleic acids of a HBsAg mRNA sequence.

In some embodiments, the inhibitory nucleic acid comprises at least 15 consecutive nucleic acids from the nucleic acid sequence set forth in SEQ ID NO:2, SEQ ID NO:5, or SEQ ID NO:6.

In some embodiments, the inhibitory nucleic acid comprises the nucleic acid sequence ACCUCUGCCUAAUCAUCUC (SEQ ID NO:4).

In some embodiments, inhibitory nucleic acid comprises between 15 and 25 nucleic acids in length.

In some embodiments, the inhibitory nucleic acid comprises an antisense oligonucleotide, a short interfering RNA (siRNA), or a short hairpin RNA (shRNA).

In some embodiments, the inhibitory nucleic acid is single-stranded or double-stranded.

In some embodiments, the inhibitory nucleic acid comprises one or more modifications.

In some embodiments, the bulevirtide, or a pharmaceutically acceptable salt thereof, and the inhibitory nucleic acid targeting hepatitis B virus (HBV), are administered simultaneously.

In some embodiments, the bulevirtide, or a pharmaceutically acceptable salt thereof, and the inhibitory nucleic acid targeting hepatitis B virus (HBV), are administered sequentially.

In some embodiments, the bulevirtide, or a pharmaceutically acceptable salt thereof, is administered subcutaneously.

In some embodiments, the bulevirtide, or a pharmaceutically acceptable salt thereof, is administered subcutaneously at a dosage of about 1 mg to about 10 mg, for example, about 1 mg, about 2 mg, about 3 mg, about 4 mg, about 5 mg, about 6 mg, about 7 mg, about 8 mg, about 9 mg, or about 10 mg.

In some embodiments, the bulevirtide, or a pharmaceutically acceptable salt thereof, is administered subcutaneously at a dosage of about 1 mg to about 5 mg. In some embodiments, the bulevirtide, or a pharmaceutically acceptable salt thereof, is administered subcutaneously at a dosage of about 1 mg to about 3 mg. In some embodiments, the bulevirtide, or a pharmaceutically acceptable salt thereof, is administered subcutaneously at a dosage of about 2 mg.

In some embodiments, the bulevirtide is bulevirtide acetate.

In some embodiments, the bulevirtide acetate is administered subcutaneously at a dosage of about 1 mg to about 10 mg, for example, about 1 mg, about 2 mg, about 3 mg, about 4 mg, about 5 mg, about 6 mg, about 7 mg, about 8 mg, about 9 mg, or about 10 mg.

In some embodiments, the bulevirtide acetate is administered subcutaneously at a dosage of about 1 mg to about 5 mg. In some embodiments, the bulevirtide acetate is administered subcutaneously at a dosage of about 1 mg to about 3 mg. In some embodiments, the bulevirtide acetate is administered subcutaneously at a dosage of about 2 mg.

As used herein, the singular forms “a” and “an”, and “the” include plural referents unless the context clearly dictates otherwise. Thus, e.g., reference to “the compound” includes a plurality of such compounds and reference to “the assay” includes reference to one or more assays, and so forth.

Unless defined otherwise, all technical and scientific terms used herein have the same meaning as commonly understood by one of ordinary skill in the art.

Except as expressly defined otherwise, the present disclosure includes all tautomers of compounds detailed herein, even if only one tautomer is expressly represented (e.g., both tautomeric forms are intended and described by the presentation of one tautomeric form where a pair of two tautomers may exist). For example, if reference is made to a compound containing an amide (e.g., by structure or chemical name), it is understood that the corresponding imidic acid tautomer is included by this disclosure and described the same as if the amide were expressly recited either alone or together with the imidic acid. Where more than two tautomers may exist, the present disclosure includes all such tautomers even if only a single tautomeric form is depicted by chemical name and/or structure.

It is understood by one skilled in the art that this disclosure also includes any compound disclosed herein (e.g., bulevirtide, or a pharmaceutically acceptable salt thereof, and/or inhibitory nucleic acid targeting hepatitis B virus (HBV)) that may be enriched at any or all atoms above naturally occurring isotopic ratios with one or more isotopes such as, but not limited to, deuterium (2H or D).

Disclosed are also compounds in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds may increase resistance to metabolism, and thus may be useful for increasing the half-life of the compounds when administered to a mammal. See, e.g., Foster, “Deuterium Isotope Effects in Studies of Drug Metabolism”, Trends Pharmacol. Sci., 5(12):524-527 (1984). Such compounds are synthesized by means well known in the art, for example by employing starting materials in which one or more hydrogen atoms have been replaced by deuterium.

Examples of isotopes that can be incorporated into the disclosed compounds also include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁷O, ¹⁸O, ³¹P, ³²P, ³⁵S, ¹⁸F, ³⁶Cl, ¹²³I, and ¹²⁵I respectively. Substitution with positron emitting isotopes, such as ¹¹C, ¹⁸F, ¹⁵⁰ and ¹³N, can be useful in Positron Emission Topography (PET) studies for examining substrate receptor occupancy. An isotopically-labeled compound of Formula I can generally be prepared by conventional techniques known to those skilled in the art or by processes analogous to those described in the Examples as set out below using an appropriate isotopically-labeled reagent in place of the non-labeled reagent previously employed.

Additionally, in some embodiments, isotopically-labeled compounds of Formula I can generally be prepared by conventional techniques known to those skilled in the art or by processes analogous to those described in the Examples as set out below using an appropriate isotopically-labeled reagent in place of the non-labeled reagent previously employed.

Compounds described herein may have chiral centers and/or geometric isomeric centers (E- and Z-isomers), and it is to be understood that all such optical, enantiomeric, diastereoisomeric and geometric isomers are encompassed. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s). As used herein, “scalemic mixture” is a mixture of stereoisomers at a ratio other than 1:1.

The combination of bulevirtide, or a pharmaceutically acceptable salt thereof, and inhibitory nucleic acid targeting hepatitis B virus (HBV), of the present disclosure may be administered to an individual in accordance with an effective dosing regimen for a desired period of time or duration, such as at least about one month, at least about 2 months, at least about 3 months, at least about 6 months, or at least about 12 months or longer.

The specific dose level of the combination of the present disclosure for any particular subject will depend upon a variety of factors including the activity of the specific compound employed, the age, body weight, general health, sex, diet, time of administration, route of administration, and rate of excretion, and the severity of the particular disease in the subject undergoing therapy. For example, a dosage may be expressed as a number of milligrams of a compound provided herein, or a pharmaceutically acceptable salt thereof, per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The dosage may also be described as a total amount of a compound described herein, or a pharmaceutically acceptable salt thereof, administered per dose. The dosage or dosing frequency of the combination of the present disclosure may be adjusted over the course of the treatment, based on the judgment of the administering physician.

The combination of the present disclosure may be administered to an individual (e.g., a human) in a therapeutically effective amount. In some embodiments, the combination is administered once daily, once weekly, once monthly, once every two months, once every three months, or once every six months. In some embodiments, the combination is administered once daily. In some embodiments, the combination is administered once weekly. In some embodiments, the combination is administered once monthly. In some embodiments, the combination is administered once every two months. In some embodiments, the combination is administered once every three months. In some embodiments, the combination is administered once every six months.

A single dose of the combination can be administered hourly, daily, weekly, or monthly. For example, a single dose can be administered once every 1 hour, 2, 3, 4, 6, 8, 12, 16 or once every 24 hours. A single dose can also be administered once every 1 day, 2, 3, 4, 5, 6, or once every 7 days. A single dose can also be administered once every 1 week, 2, 3, or once every 4 weeks.

The frequency of dosage of the combination of the present disclosure will be determined by the needs of the individual patient. Administration of the combination continues for as long as necessary to treat the infection, including an HDV infection, or any other indication described herein.

Administration can be intermittent, with a period of several or more days during which a patient receives a daily dose of the combination of the present disclosure, followed by a period of several or more days during which a patient does not receive a daily dose of the combination. For example, a patient can receive a dose of the combination, every other day, or three times per week. Again by way of example, a patient can receive a dose of the combination each day for a period of from 1 to 14 days, followed by a period of 7 to 21 days during which the patient does not receive a dose of the combination followed by a subsequent period (e.g., from 1 to 14 days) during which the patient again receives a daily dose of the combination. Alternating periods of administration of the combination, followed by non-administration of the combination, can be repeated as clinically required to treat the patient.

### Pharmaceutical Formulations

In some embodiments, the methods provided herein comprise administering to the patient:
i) a pharmaceutical composition comprising bulevirtide, or a pharmaceutically acceptable salt thereof, and one or more pharmaceutically acceptable excipients; and
ii) a pharmaceutical composition comprising an inhibitory nucleic acid targeting HBV (e.g., an inhibitory nucleic acid targeting HBc, HBx, HBsAg, or a combination of any of these), and one or more pharmaceutically acceptable excipients.

In some embodiments, the pharmaceutical composition comprising the bulevirtide, or a pharmaceutically acceptable salt thereof, and one or more pharmaceutically acceptable excipients; and the pharmaceutical composition comprising the inhibitory nucleic acid targeting HBV (e.g., an inhibitory nucleic acid targeting HBc, HBx, HBsAg, or a combination of any of these), and one or more pharmaceutically acceptable excipients, are administered simultaneously.

In some embodiments, the pharmaceutical composition comprising the bulevirtide, or a pharmaceutically acceptable salt thereof, and one or more pharmaceutically acceptable excipients; and the pharmaceutical composition comprising the inhibitory nucleic acid targeting HBV (e.g., an inhibitory nucleic acid targeting HBc, HBx, HBsAg, or a combination of any of these), and one or more pharmaceutically acceptable excipients, are administered sequentially.

In some embodiments, the inhibitory nucleic acid targets HBc.

In some embodiments, the inhibitory nucleic acid targets HBx.

In some embodiments, the inhibitory nucleic acid targets HBsAg.

In some embodiments, the inhibitory nucleic acid targets any combination of HBc, HBx, and HBsAg.

The pharmaceutical compositions disclosed herein can be prepared by methodologies well known in the pharmaceutical art. For example, in certain embodiments, a pharmaceutical composition intended to be administered by injection can prepared by combining a compound of the disclosure with sterile, distilled water so as to form a solution. In some embodiments, a surfactant is added to facilitate the formation of a homogeneous solution or suspension. Surfactants are compounds that non-covalently interact with the compound of the disclosure so as to facilitate dissolution or homogeneous suspension of the compound in the aqueous delivery system.

Administration of the combination of the disclosure can be carried out via any of the accepted modes of administration of agents for serving similar utilities. The pharmaceutical compositions of the disclosure can be prepared by combining a compound of the disclosure, or a pharmaceutically acceptable salt thereof, with an appropriate pharmaceutically acceptable carrier and, in specific embodiments, are formulated into preparations in solid, semi solid, liquid or gaseous forms, such as tablets, capsules, powders, granules, ointments, solutions, suppositories, injections, inhalants, gels, microspheres, and aerosols. Exemplary routes of administering such pharmaceutical compositions include, without limitation, oral, topical, transdermal, inhalation, parenteral, sublingual, buccal, rectal, vaginal, and intranasal. Pharmaceutical compositions of the disclosure are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the disclosure in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of a compound of the disclosure, or a pharmaceutically acceptable salt thereof, for treatment of a disease or condition of interest in accordance with the teachings described herein.

Pharmaceutical compositions containing an active ingredient may be in any form suitable for the intended method of administration. In some embodiments, the compound or salt is prepared according to one or more of the processes described herein.

“Pharmaceutically acceptable” refers to compounds, salts, compositions, dosage forms and other materials which are useful in preparing a pharmaceutical composition that is suitable for veterinary or human pharmaceutical use.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

Pharmaceutical compositions provided herein may be prepared with conventional carriers (e.g., inactive ingredient or excipient material) which may be selected in accord with ordinary practice. For example, tablets may contain excipients including glidants, fillers, binders and the like. Aqueous compositions may be prepared in sterile form, and when intended for delivery by other than oral administration generally may be isotonic. All compositions may optionally contain excipients such as those set forth in the Rowe et al, Handbook of Pharmaceutical Excipients, 5^{th} edition, American Pharmacists Association, 1986.

In some embodiments, a first pharmaceutical composition disclosed herein comprises bulevirtide, or a pharmaceutically acceptable salt thereof, and one or more pharmaceutically acceptable excipients.

In some embodiments, a first pharmaceutical composition disclosed herein comprises bulevirtide or an acetic acid salt of bulevirtide (i.e., bulevirtide acetate), and one or more pharmaceutically acceptable excipients.

In some embodiments, the pharmaceutical composition comprises about 1 mg to about 5 mg of the bulevirtide, or a pharmaceutically acceptable salt thereof, and one or more pharmaceutically acceptable excipients, for example, about 1 mg, about 2 mg, about 3 mg, about 4 mg, or about 5 mg of the bulevirtide, or a pharmaceutically acceptable salt thereof. In some embodiments, the pharmaceutical composition comprises about 2 mg of the bulevirtide, or a pharmaceutically acceptable salt thereof, and one or more pharmaceutically acceptable excipients. In some embodiments, the pharmaceutical composition comprises about 2 mg on a free base basis of bulevirtide acetate, and one or more pharmaceutically acceptable excipients.

In some embodiments, the pharmaceutical composition comprising the bulevirtide, or a pharmaceutically acceptable salt thereof, is an injectable solution. In some embodiments, the pharmaceutical composition comprising the bulevirtide, or a pharmaceutically acceptable salt thereof, is an injectable solution suitable for subcutaneous administration to the patient.

In some embodiments, the injectable solution comprises about 1 mg to about 5 mg of the bulevirtide, or a pharmaceutically acceptable salt thereof, and one or more pharmaceutically acceptable excipients, for example, about 1 mg, about 2 mg, about 3 mg, about 4 mg, or about 5 mg of the bulevirtide, or a pharmaceutically acceptable salt thereof. In some embodiments, the injectable solution comprises about 2 mg of the bulevirtide, or a pharmaceutically acceptable salt thereof, and one or more pharmaceutically acceptable excipients. In some embodiments, the injectable solution comprises about 2 mg on a free base basis of bulevirtide acetate, and one or more pharmaceutically acceptable excipients.

In some embodiments, the injectable solution comprises one or more pharmaceutically acceptable excipients selected from sodium carbonate, sodium bicarbonate, and mannitol; or any combination thereof.

In some embodiments, the injectable solution comprises bulevirtide, or a pharmaceutically acceptable salt thereof, sodium carbonate, sodium bicarbonate, and mannitol. In some embodiments, the injectable solution comprises bulevirtide acetate, sodium carbonate, sodium bicarbonate, and mannitol. In some embodiments, the injectable solution comprises about 2 mg on a free base basis of bulevirtide acetate, sodium carbonate, sodium bicarbonate, and mannitol.

In some embodiments, the injectable solution comprises:
about 2 mg on a free base basis of bulevirtide acetate;
about 0.1% w/w to about 0.5% w/w of sodium carbonate;
about 3.5% w/w to about 4.0% w/w of sodium bicarbonate; and
about 90% w/w to about 95% w/w of mannitol.

In some embodiments, the injectable solution comprises:
about 2 mg on a free base basis of bulevirtide acetate;
about 0.25% w/w of sodium carbonate;
about 3.7% w/w of sodium bicarbonate; and
about 92% w/w of mannitol.

In some embodiments, the injectable solution provided herein further comprises one or more buffering agents and a solvent. In some embodiments, the one or more buffering agents comprise sodium hydroxide and hydrochloric acid. In some embodiments, the solvent comprises water.

In some embodiments, a second pharmaceutical composition disclosed herein comprises an inhibitory nucleic acid targeting HBV (e.g., an inhibitory nucleic acid targeting HBc, HBx, HBsAg, or a combination of any of these), and one or more pharmaceutically acceptable excipients.

In some embodiments, the inhibitory nucleic acid targeting HBV is administered by intravenous or subcutaneous administration (see e.g., International Patent Application Publication No.: WO2023225598A2, the disclosure of which is incorporated herein by reference in its entirety). In some embodiments, the inhibitory nucleic acid targeting HBV is administered by intravenous administration. In some embodiments, the inhibitory nucleic acid targeting HBV is administered by subcutaneous administration.

In some embodiments, the pharmaceutical composition disclosed herein comprising an inhibitory nucleic acid targeting HBV (e.g., an inhibitory nucleic acid targeting HBc, HBx, HbsAg, or a combination of any of these), and one or more pharmaceutically acceptable excipients, comprises N-acetylgalactosamine (GALNAC), or a derivative thereof. In some embodiments, the N-acetylgalactosamine (GALNAC), or a derivative thereof, enhances liver targeting of the pharmaceutical composition.

### Methods of Use

The present application further provides a method of inhibiting or treating an HBV and/or HDV infection in a patient in need thereof, comprising administering to a patient (e.g., a patient in need thereof), bulevirtide, or a pharmaceutically acceptable salt thereof, and an inhibitory nucleic acid targeting HBV (e.g., an inhibitory nucleic acid targeting HBc, HBx, HBsAg, or a combination of any of these).

The present application further provides a method of inhibiting or treating an HDV infection in a patient in need thereof, comprising administering to a patient (e.g., a patient in need thereof), bulevirtide, or a pharmaceutically acceptable salt thereof, and an inhibitory nucleic acid targeting HBV (e.g., an inhibitory nucleic acid targeting HBc, HBx, HBsAg, or a combination of any of these).

In some embodiments, the patient has been identified as having hepatitis B. In some embodiments, the patient has been identified as having compensated liver disease (e.g, compensated cirrhosis). As used herein, the terms “compensated liver disease” or “compensated cirrhosis” refer to asymptomatic liver disease (e.g., asymptomatic cirrhosis). In some embodiments, the patient identified as having compensated liver disease does not exhibit one or more symptoms of the liver disease, including, but not limited to, ascites, variceal hemorrhage, hepatic encephalopathy, and jaundice. In some embodiments, the patient identified as having compensated liver disease does not exhibit symptoms of the liver disease, including, but not limited to, ascites, variceal hemorrhage, hepatic encephalopathy, and jaundice.

The present application further provides a method of preventing a primary HBV and/or HDV infection in a patient in need thereof, comprising administering to the patient bulevirtide, or a pharmaceutically acceptable salt thereof, and an inhibitory nucleic acid targeting HBV (e.g., an inhibitory nucleic acid targeting HBc, HBx, HBsAg, or a combination of any of these).

The present application further provides a method of preventing an HDV infection in a patient in need thereof, comprising administering to the patient bulevirtide, or a pharmaceutically acceptable salt thereof, and an inhibitory nucleic acid targeting HBV (e.g., an inhibitory nucleic acid targeting HBc, HBx, HBsAg, or a combination of any of these).

The present application further provides methods of treating and/or preventing chronic hepatitis B and/or D in a patient in need thereof, comprising administering to the patient bulevirtide, or a pharmaceutically acceptable salt thereof, and an inhibitory nucleic acid targeting HBV (e.g., an inhibitory nucleic acid targeting HBc, HBx, HBsAg, or a combination of any of these).

The present application further provides methods of treating and/or preventing chronic hepatitis D in a patient in need thereof, comprising administering to the patient bulevirtide, or a pharmaceutically acceptable salt thereof, and an inhibitory nucleic acid targeting HBV (e.g., an inhibitory nucleic acid targeting HBc, HBx, HBsAg, or a combination of any of these).

The present application further provides bulevirtide, or a pharmaceutically acceptable salt thereof, and an inhibitory nucleic acid targeting HBV (e.g., an inhibitory nucleic acid targeting HBc, HBx, HBsAg, or a combination of any of these), for use in any of the methods described herein.

The present application further provides bulevirtide, or a pharmaceutically acceptable salt thereof, and an inhibitory nucleic acid targeting HBV (e.g., an inhibitory nucleic acid targeting HBc, HBx, HBsAg, or a combination of any of these), for the preparation of a medicament for use in any of the methods described herein.

The terms “patient” and “patients” refers to humans, domestic animals (e.g., dogs and cats), farm animals (e.g., cattle, horses, sheep, goats and pigs), laboratory animals (e.g., mice, rats, hamsters, guinea pigs, pigs, rabbits, dogs, and monkeys), and the like. In some embodiments, the patient is a human patient.

As used herein, “treatment” or “treating” is an approach for obtaining beneficial or desired results. For purposes of the present disclosure, beneficial or desired results include, but are not limited to, alleviation of a symptom and/or diminishment of the extent of a symptom and/or preventing a worsening of a symptom associated with a disease or condition. In one embodiment, “treatment” or “treating” includes one or more of the following: a) inhibiting the disease or condition (e.g., decreasing one or more symptoms resulting from the disease or condition, and/or diminishing the extent of the disease or condition); b) slowing or arresting the development of one or more symptoms associated with the disease or condition (e.g., stabilizing the disease or condition, delaying the worsening or progression of the disease or condition); and/or c) relieving the disease or condition, e.g., causing the regression of clinical symptoms, ameliorating the disease state, delaying the progression of the disease, increasing the quality of life, and/or prolonging survival.

As used herein, “prevention” or “preventing” refers to a regimen that protects against the onset of the disease or disorder such that the clinical symptoms of the disease do not develop. Thus, “prevention” relates to administration of a therapy (e.g., administration of a therapeutic substance) to a patient before signs of the disease are detectable in the patient (e.g., administration of a therapeutic substance to a patient in the absence of detectable infectious agent (e.g., virus) in the patient). The patient may be an individual at risk of developing the disease or disorder, such as an individual who has one or more risk factors known to be associated with development or onset of the disease or disorder. Thus, the terms “preventing HBV infection” and “preventing HDV infection” refer to administering to a patient who does not have a detectable HBV or HDV infection an anti-HBV or HDV therapeutic substance (e.g. bulevirtide, or a pharmaceutically acceptable salt thereof, and an inhibitory nucleic acid targeting hepatitis B virus (HBV), as described herein). It is understood that the patient for preventative bulevirtide therapy may be an individual at risk of contracting the HBV and/or HDV virus. Further, it is understood that prevention may not result in complete protection against onset of the disease or disorder. In some instances, prevention includes reducing the risk of developing the disease or disorder. The reduction of the risk may not result in complete elimination of the risk of developing the disease or disorder.

As used herein, an “at risk” individual is an individual who is at risk of developing a condition to be treated. An individual “at risk” may or may not have detectable disease or condition, and may or may not have displayed detectable disease prior to the treatment of methods described herein. “At risk” denotes that an individual has one or more so-called risk factors, which are measurable parameters that correlate with development of a disease or condition and are known in the art. An individual having one or more of these risk factors has a higher probability of developing the disease or condition than an individual without these risk factor(s).

As used herein, the term “therapeutically effective amount” or “effective amount” refers to an amount that is effective to elicit the desired biological or medical response, including the amount of a compound that, when administered to a patient for treating a disease, is sufficient to affect such treatment for the disease or to an amount that is effective to protect against the contracting or onset of a disease. The effective amount will vary depending on the compound, the disease, and its severity and the age, weight, etc., of the patient to be treated. The effective amount can include a range of amounts. As is understood in the art, an effective amount may be in one or more doses, i.e., a single dose or multiple doses may be required to achieve the desired treatment outcome. An effective amount may be considered in the context of administering one or more therapeutic agents, and a single agent may be considered to be given in an effective amount if, in conjunction with one or more other agents, a desirable or beneficial result may be or is achieved. Suitable doses of any co-administered compounds may optionally be lowered due to the combined action (e.g., additive or synergistic effects) of the compounds.

### Combination Therapies

In some embodiments, pharmaceutical compositions including an agent(s) of the present disclosure, or a pharmaceutically acceptable salt thereof, in combination with one or more (e.g., one, two, three, four, one or two, one to three, or one to four) additional therapeutic agents, and a pharmaceutically acceptable excipient are provided.

In some embodiments, kits including an agent(s) of the present disclosure, or a pharmaceutically acceptable salt thereof, in combination with one or more (e.g., one, two, three, four, one or two, one to three, or one to four) additional therapeutic agents are provided.

In some embodiments, an agent(s) of the present disclosure, or a pharmaceutically acceptable salt thereof, is combined with one, two, three, four or more additional therapeutic agents. In some embodiments, an agent(s) of the present disclosure, or a pharmaceutically acceptable salt thereof, is combined with two additional therapeutic agents. In some embodiments, an agent(s) of the present disclosure, or a pharmaceutically acceptable salt thereof, is combined with three additional therapeutic agents. In some embodiments, an agent(s) of the present disclosure, or a pharmaceutically acceptable salt thereof, is combined with four additional therapeutic agents. The one, two, three, four or more additional therapeutic agents can be different therapeutic agents selected from the same class of therapeutic agents, and/or they can be selected from different classes of therapeutic agents.

In some embodiments, when an agent(s) of the present disclosure is combined with one or more additional therapeutic agents as described herein, the components of the composition are administered as a simultaneous or sequential regimen. When administered sequentially, the combination may be administered in two or more administrations.

Co-administration of an agent(s) disclosed herein with one or more additional therapeutic agents generally refers to simultaneous or sequential administration of an agent disclosed herein and one or more additional therapeutic agents, such that therapeutically effective amounts of each agent are present in the body of the patient.

Co-administration includes administration of unit dosages of the agent(s) disclosed herein before or after administration of unit dosages of one or more additional therapeutic agents. The agent(s) disclosed herein may be administered within seconds, minutes, or hours of the administration of one or more additional therapeutic agents. For example, in some embodiments, a unit dose of an agent(s) disclosed herein is administered first, followed within seconds or minutes by administration of a unit dose of one or more additional therapeutic agents. Alternatively, in some embodiments, a unit dose of one or more additional therapeutic agents is administered first, followed by administration of a unit dose of an agent(s) disclosed herein within seconds or minutes. In some embodiments, a unit dose of an agent(s) disclosed herein is administered first, followed, after a period of hours (e.g., 1-12 hours), by administration of a unit dose of one or more additional therapeutic agents. In some embodiments, a unit dose of one or more additional therapeutic agents is administered first, followed, after a period of hours (e.g., 1-12 hours), by administration of a unit dose of an agent(s) disclosed herein.

In some embodiments, an agent(s) of the present disclosure is combined with one or more additional therapeutic agents in a unitary dosage form for simultaneous administration to a patient, for example as a solid dosage form for oral administration.

In some embodiments, an agent(s) of the present disclosure is combined with one, two, three, four or more additional therapeutic agents selected from HDV combination drugs, HBV vaccines, DNA polymerase inhibitors, interferon alpha receptor ligands, hepatitis b surface antigen (HBsAg) inhibitors, HDV/HBV viral entry inhibitors, sodium bile acid cotransporter inhibitors, gene expression inhibitors (eg siRNA), farnesoid X receptor agonists, anti-HBV antibodies, anti-PD-1 antibodies, HIV protease inhibitors, nucleoside reverse transcriptase inhibitors and other HBV drugs.

In some embodiments, the additional therapeutic agent is a DNA polymerase inhibitor, such as fosclevudine alafenamide, entecavir.

In some embodiments, the additional therapeutic agent is an interferon alpha receptor ligand, such as peginterferon alfa-2b, peginterferon alfa-2a, interferon, ropeginterferon-alfa-2b, peginterferon lambda-1a (BMS-914143).

In some embodiments, the additional therapeutic agent is a hepatitis B surface antigen (HBsAg) inhibitor, such as REP-9 (REP 2139-Mg), BJT-778, BM-012.

In some embodiments, the additional therapeutic agent is an HDV/HBV viral entry inhibitor, such as hepalatide, HH-003, AB-543, AB-6250, and HH-006.

In some embodiments, the additional therapeutic agent is a sodium bile acid cotransporter inhibitor such as A-2342 and AB-6250.

In some embodiments, the additional therapeutic agent is a gene expression inhibitor, such as JNJ-3989, VIR-2218 (ALN-HBV-02), AB-729, RG6346, Bepiprovirsen.

In some embodiments, the additional therapeutic agent is a HBV antibody, such as VIR-3434.

In some embodiments, the additional therapeutic agent is an anti-PD-1 antibody, such as nivolumab.

In some embodiments, the additional therapeutic agent is an HIV protease inhibitor, such as ritonavir.

In some embodiments, the additional therapeutic agent is a nucleoside reverse transcriptase inhibitor, such as tenofovir disoproxil fumarate.

In some embodiments, an agent(s) of the present disclosure, is combined with one, two, three, four or more additional therapeutic agents selected from HBV combination drugs, HBV vaccines, HBV DNA polymerase inhibitors, immunomodulators, toll-like receptor (TLR) modulators, interferon alpha receptor ligands, hepatitis b core antigen (HBcAg) inhibitors, hepatitis b surface antigen (HBsAg) inhibitors, hepatitis B virus X interacting protein inhibitor, cytotoxic T-lymphocyte-associated protein 4 (CTLA4) inhibitors, cyclophilin inhibitors, HBV viral entry inhibitors, antisense oligonucleotide targeting viral mRNA, short interfering RNAs (siRNA) and ddRNAi, endonuclease modulators, ribonucelotide reductase inhibitors, HBV E antigen inhibitors, covalently closed circular DNA (cccDNA) inhibitors, farnesoid X receptor agonists, HBV antibodies, Complement C5 factor modulator, CCR2 chemokine antagonists, Caspase-9 stimulator, CD3 modulator, thymosin agonists, cytokines, nucleoprotein modulators, retinoic acid-inducible gene 1 stimulators, NOD2 stimulators, MEKKK 1 protein kinase (HPK1 checkpoint) inhibitors, nucleic acid polymers (e.g., NAPs and STOPS), PD-1 inhibitors, PD-L1 inhibitors, synthetic antiviral peptoids, HBV replication inhibitors, gene therapy and cell therapy, gene editors, CAR-T cell therapy, TCR-T cell therapy, and other HBV drugs.

In some embodiments, an agent(s) as described herein, may be used or combined with one or more of a chemotherapeutic agent, an immunomodulator, an immunotherapeutic agent, a therapeutic antibody, a therapeutic vaccine, a bispecific antibody and “antibody-like” therapeutic protein (such as DARPins®, anti-pMHC TCR-like antibodies, DARTs®, Duobodies®, Bites®, XmAbs®, TandAbs®, Fab derivatives), an antibody-drug conjugate (ADC), gene modifiers or gene editors (such as CRISPR Cas9, zinc finger nucleases, homing endonucleases, homing meganucleases (e.g., ARCUS), synthetic nucleases, TALENs), cell therapies such as CAR-T (chimeric antigen receptor T-cell), and TCR-T (an engineered T cell receptor) agent or any combination thereof.

In some embodiments, an agent(s) as described herein, is combined with one, two, three, four or more additional therapeutic agents, e.g., as 3-dioxygenase (IDO) inhibitors, apolipoprotein A1 modulator, arginase inhibitors, B- and T-lymphocyte attenuator inhibitors, Bruton's tyrosine kinase (BTK) inhibitors, CCR2 chemokine antagonist, CD137 inhibitors, CD160 inhibitors, CD305 inhibitors, CD4 agonist and modulator, compounds targeting hepatitis B core antigen (HBcAg), core protein allosteric modulators, covalently closed circular DNA (cccDNA) inhibitors, cyclophilin inhibitors, cytotoxic T-lymphocyte-associated protein 4 (ipi4) inhibitors, DNA polymerase inhibitor, endonuclease modulators, epigenetic modifiers, Farnesoid X receptor agonists, free fatty acid (Ffa) receptor 2 (Ffar2; PR43) agonists, free fatty acid (Ffa) receptor 3 (Ffar3; GPR441) agonists, HBV DNA polymerase inhibitors, HBV replication inhibitors, HBV RNAse inhibitors, HBV viral entry inhibitors, HBx inhibitors, Hepatitis B large envelope protein inhibitor, Hepatitis B large envelope protein stimulator, Hepatitis B structural protein modulator, hepatitis B surface antigen (HBsAg) inhibitors, hepatitis B surface antigen (HBsAg) secretion or assembly inhibitors, hepatitis B virus E antigen inhibitors, hepatitis B virus replication inhibitors, Hepatitis virus structural protein inhibitor, HIV-1 reverse transcriptase inhibitor, Hyaluronidase inhibitor, inhibitor of apoptosis proteins family proteins (IAPs) inhibitors, IL-2 agonist, IL-7 agonist, immunomodulators, indoleamine-2 inhibitors, inhibitors of ribonucleotide reductase, Interleukin-2 ligand, ipi4 inhibitors, lysine demethylase inhibitors, histone demethylase inhibitors, KDM1 inhibitors, KDM5 inhibitors, killer cell lectin-like receptor subfamily G member 1 inhibitors, lymphocyte-activation gene 3 inhibitors, lymphotoxin beta receptor activators, modulators of Axl, modulators of B7-H3, modulators of B7-H4, modulators of CD160, modulators of CD161, modulators of CD27, modulators of CD47, Non canonical RNA polymerase PAPD5 inhibitors. Non canonical RNA polymerase PAPD7 inhibitors, modulators of CD70, modulators of GITR, modulators of HEVEM, modulators of ICOS, modulators of Mer, modulators of NKG2A, modulators of NKG2D, modulators of OX40, modulators of SIRPalpha, modulators of TIGIT, modulators of Tim-4, modulators of Tyro, Na+-taurocholate cotransporting polypeptide (NTCP) inhibitors, natural killer cell receptor 2B4 inhibitors, NOD2 gene stimulator, Nucleoprotein inhibitor, nucleoprotein modulators, OX-40 receptor agonist, PD-1 inhibitors, PD-L1 inhibitors, peptidylprolyl isomerase inhibitor, phosphatidylinositol-3 kinase (PI3K) inhibitors, Retinoic acid-inducible gene 1 stimulator, Reverse transcriptase inhibitor, Ribonuclease inhibitor, RNA DNA polymerase inhibitor, SLC10A1 gene inhibitor, SMAC mimetics, Src tyrosine kinase inhibitor, stimulator of interferon gene (STING) agonists, stimulators of NOD1, T cell surface glycoprotein CD28 inhibitor, T-cell surface glycoprotein CD8 modulator, Thymosin agonist, Thymosin alpha 1 ligand, Tim-3 inhibitors, TLR-3 agonists, TLR-7 agonists, TLR-7 modulators, TLR-8 modulators, TLR-9 agonists, TLR9 agonists or gene stimulator, toll-like receptor (TLR) modulators, viral ribonucleotide reductase inhibitors, and combinations thereof.

In some embodiments, an agent(s) of the present disclosure is combined with one, two, three, or four additional therapeutic agents as disclosed herein.

### HBV Combination Drugs

i. Antiviral Agents

### HBV DNA Polymerase Inhibitors

Examples of HBV DNA polymerase inhibitors include, but are not limited to, adefovir (HEPSERA®), emtricitabine (EMTRIVA®), emtricitabine+tenofovir disoproxil fumarate (TRUVADA®), tenofovir disoproxil fumarate (VIREAD®), tenofovir alafenamide, tenofovir, tenofovir disoproxil, tenofovir alafenamide fumarate, tenofovir alafenamide hemifumarate, tenofovir dipivoxil, tenofovir dipivoxil fumarate, tenofovir octadecyloxyethyl ester, CMX-157, tenofovir exalidex, besifovir, entecavir (BARACLUDE®), entecavir maleate, telbivudine (TYZEKA®), filocilovir, pradefovir, clevudine, ribavirin, lamivudine (EPIVIR-HBV®), phosphazide, famciclovir, fusolin, metacavir, fosclevudine alafenamide (ATI-2173), telbivudine, SNC-019754, FMCA, AGX-1009, AR-II-04-26, HIP-1302, tenofovir disoproxil aspartate, tenofovir disoproxil orotate, tenofovir disoproxil phosphate, tenofovir amibufenamide fumarate, AiB-001, hepenofovir fumarate, tenofovir fumarate, KL-210122, and HS-10234.

### Hepatitis B Surface Antigen (HBsAg) Inhibitors

Examples of HBsAg inhibitors include, but are not limited to, AK-074, HBF-0259, GP-605, PBHBV-001, PBHBV-2-15, PBHBV-2-1, REP-9AC, REP-9C, REP-9, REP-2139, REP-2139-Ca, REP-2165-Mg, REP-2055, REP-2163, REP-2165, REP-2053, REP-2031, REP-006, CB-07, GST-HG121, GST-HG131, BTJ-778, NWP-1080, and REP-9AC′.

Examples of HBsAg secretion inhibitors include, but are not limited to, BM601, GST-HG-131, AB-452, and ALG-010093.

### HBV Viral Entry Inhibitors

Examples of HBV viral entry inhibitors include, but are not limited to, AB-543, HH-003, and HH-006.

### Sodium Bile Acid Cotransporter (NTCP) Inhibitors

Examples of sodium bile acid cotransporter inhibitors include, but are not limited to A2342, L-47, PRX-202, CIM-212930, IPN-60260, AB6250.

### Hepatitis B Large Envelope Protein Inhibitors

Examples of Hepatitis B large envelope protein inhibitors include, but are not limited to, EDP-721, KW-027, GP-605, GST-HG-121, ALG-010093, ALG-01013, BJT-778, and SAG-282.

Antisense Oligonucleotide Targeting Viral mRNA

Examples of antisense oligonucleotide targeting viral mRNA include, but are not limited to, ISIS-HBVRx, Bepirovirsen, IONIS-HBV-LRx, IONIS-GSK6-LRx, GSK-3389404, BNC-1701, SB-527 and RG-6004.

Short Interfering RNAs (siRNA) and ddRNAi

Examples of siRNA include, but are not limited to, TKM-HBV (TKM-HepB), ALN-HBV, SR-008, HepB-nRNA, ARC-520, ARC-521, ARB-1740, ARB-1467, AB-729, RG-6084 (PD-L1), RG-6217, JNJ-3989 (ARO-HBV), STP-155G, STSG-0002, ALG-010133, ALG-020755, ALG-125755, ALG-126081, ALG-126101, SR-016 (RBD1016), TQA-3729, Hepbarna (BB-103), KW-040, elebsiran (VIR-2218), xalnesiran (RG-6346), ALG-125097, ALG-ASO, LUNAR-HBV, DCR-HBVS (DCR-5219), OLX-703A, Kylo-02, Kylo-04, HT-101, AHB-137, and STP-145G.

Examples of DNA-directed RNA interference (ddRNAi) include BB-HB-331.

### Non-Nucleoside Reverse Transcriptase Inhibitors

Examples of non-nucleoside reverse transcriptase inhibitors (NNRTIs) include, but are not limited to, the compounds disclosed in WO2018118826 (Merck), WO2018080903 (Merck), WO2018119013 (Merck), WO2017100108 (Idenix), WO2017027434 (Merck), WO2017007701 (Merck), WO2008005555 (Gilead).

### HBV Replication Inhibitors

Examples of hepatitis B virus replication inhibitors include, but are not limited to, ALG-000286, ASN-008, KW-034, GP-31502, isothiafludine, IQP-HBV, RM-5038, and Xingantie.

### HIV-1 Reverse Transcriptase Inhibitors

Examples of HIV-1 reverse transcriptase inhibitors include, but are not limited to, 2,5,6-substituted pyrimidone derivative (HBV).

### HBV Transcript Inhibitors

Examples of HBV transcript inhibitors include, but are not limited to, BJT-628.

Non Canonical RNA Polymerase PAPD5 and PAPD7 Inhibitors Examples of non canonical RNA polymerase PAPD5 and PAPD7 inhibitors include, but are not limited to, PAPD5 and PAPD7 targeting locked nucleic acid antisense oligonucleotides (HBV infection), GSK3965193, GST-HG131, and AB-161.
Covalently Closed Circular DNA (cccDNA) Inhibitors

Examples of cccDNA inhibitors include, but are not limited to, BSBI-25, ccc-R08, and CHR-101.

### Nucleoprotein Modulators

Nucleoprotein modulators may be either HBV core or capsid protein inhibitors. Examples of nucleoprotein modulators include, but are not limited to, GS-4882, AB-423, AB-836, AT-130, ALG-001075, ALG-001024, ALG-000184, EDP-514, GLS4, NVR-1221, NVR-3778, AL-3778, BAY 41-4109, morphothiadine mesilate, ARB-168786, ARB-880, ARB-1820, GST-HG-141, bersacapavir (JNJ-379), JNJ-632, RG-7907, GST-HG-141, HEC-72702, KL-060332, AB-506, vebicorvir (ABI-H0731), ABI-H3733, ABI-4334, JNJ-440, AK-0605, HRS-5091, VNRX-9945, ABI-H2158, canocapavir (CB-HBV-001), SOC-10, SOC-11, KW-034, GLP-26, QL-007, GST-HG-141, HEC-121120, ZM-H1505R, TQA-3605, ATI-1428, ATI-1645, XTYW-001, ALG-005398, ALG-006162, ALG-000111, GST-HG141, A-204, PLM-401 and DVR-23.

Examples of capsid inhibitors include, but are not limited to, the compounds disclosed in US2018161307 (Gilead Sciences), US20140275167 (Novira Therapeutics), US20130251673 (Novira Therapeutics), US20140343032 (Roche), WO2014037480 (Roche), US20130267517 (Roche), WO2014131847 (Janssen), WO2014033176 (Janssen), WO2014033170 (Janssen), WO2014033167 (Janssen), WO2015/059212 (Janssen), WO2015118057 (Janssen), WO2015011281 (Janssen), WO2014184365 (Janssen), WO2014184350 (Janssen), WO2014161888 (Janssen), WO2013096744 (Novira), US20150225355 (Novira), US20140178337 (Novira), US20150315159 (Novira), US20150197533 (Novira), US20150274652 (Novira), US20150259324, (Novira), US20150132258 (Novira), U.S. Pat. No. 9,181,288 (Novira), WO2014184350 (Janssen), WO2013144129 (Roche), WO2017198744 (Roche), US 20170334882 (Novira), US 20170334898 (Roche), WO2017202798 (Roche), WO2017214395 (Enanta), WO2018001944 (Roche), WO2018001952 (Roche), WO2018005881 (Novira), WO2018005883 (Novira), WO2018011100 (Roche), WO2018011160 (Roche), WO2018011162 (Roche), WO2018011163 (Roche), WO2018036941 (Roche), WO2018043747 (Kyoto Univ), US20180065929 (Janssen), WO2016168619 (Indiana University), WO2016195982 (The Penn State Foundation), WO2017001655 (Janssen), WO2017048950 (Assembly Biosciences), WO2017048954 (Assembly Biosciences), WO2017048962 (Assembly Biosciences), US20170121328 (Novira), US20170121329 (Novira), H-05, HBV capsid inhibitors (Arbutus Biopharma, Chinese Academy of Sciences/Shanghai University of Traditional Chinese Medicine).

Examples of transcript inhibitors include, but are not limited to, the compounds disclosed in WO2017013046 (Roche), WO2017016960 (Roche), WO2017017042 (Roche), WO2017017043 (Roche), WO2017061466 (Toyoma chemicals), WO2016177655 (Roche), WO2016161268 (Enanta), WO2017001853 (Redex Pharma), WO2017211791 (Roche), WO2017216685 (Novartis), WO2017216686 (Novartis), WO2018019297 (Ginkgo Pharma), WO2018022282 (Newave Pharma), US20180030053 (Novartis), and WO2018045911 (Zhejiang Pharma).

### Farnesoid X Receptor Agonists

Examples of farnesoid x receptor agonists include, but are not limited to, e.g., Vonafexor (EYP001), cilofexor (GS-9674), EDP-305, MET-409, Tropifexor, AKN-083, RDX-023, BWD-100, LMB-763, INV-3, NTX-023-1, EP-024297, ASC-42, HEC-96719 and GS-8670.

### Caspase-9 Stimulators

Examples of Caspase-9 stimulators include, but are not limited to, ENOB-HB-01.

### HBV Antibodies

Examples of HBV antibodies targeting the surface antigens of the hepatitis B virus include, but are not limited to, lenvervimab (GC-1102), XTL-17, XTL-19, KN-003, IV Hepabulin SN, VIR-3434, 162 (Yangshengtang/Syneos Health), APB-A101, and fully human monoclonal antibody therapy (hepatitis B virus infection, Humabs BioMed).

Examples of HBV antibodies, including monoclonal antibodies and polyclonal antibodies, include, but are not limited to, Zutectra, Shang Sheng Gan Di, Uman Big (Hepatitis B Hyperimmune), Omri-Hep-B, Nabi-HB, Hepatect CP, HepaGam B, igantibe, Niuliva, CT-P24, EI-001, hepatitis B immunoglobulin (intravenous, pH4, HBV infection, Shanghai RAAS Blood Products), EVT-075, and Fovepta (BT-088).

Examples of fully human monoclonal antibodies include, but are not limited to, HBC-34, HT-102.

Antibodies against HBV viral peptide/major histocompatibility complex (MHC) class I (pMHC) complexes are described, e.g., in Sastry et al., J Virol. 2011 March; 85(5):1935-42 and in WO2011062562.

Examples of IgG4 monoclonal antibodies include burfiralimab.

Examples of IFN-fused anti-CD40 antibodies include type I IFN/CD40 costimulator (HBV infection).

ii. Immune Modulators

### HBV Vaccines

HBV vaccines include both prophylactic and therapeutic vaccines. Examples of HBV prophylactic vaccines include, but are not limited to, Vaxelis, Hexaxim, Heplisav, Mosquirix, DTwP-HBV vaccine, PreHevbri (Bio-Hep-B), D/T/P/HBV/M (LBVP-0101; LBVW-0101), DTwP-Hepb-Hib-IPV vaccine, Heberpenta L, DTwP-HepB-Hib, V-419, CVI-HBV-001, Tetrabhay, hepatitis B prophylactic vaccine (Advax Super D), Hepatrol-07, GSK-223192A, ENGERIX B®, recombinant hepatitis B vaccine (intramuscular, Kangtai Biological Products), recombinant hepatitis B vaccine (Hansenual polymorpha yeast, intramuscular, Hualan Biological Engineering), recombinant hepatitis B surface antigen vaccine, Bimmugen, CARG-101, Euforavac, Eutravac, anrix-DTaP—IPV-Hep B, HBAI-20, Infanrix-DtaP—IPV-Hep B-Hib, Pentabio Vaksin DTP—HB-Hib, Comvac 4, Twinrix, Euvax-B, Tritanrix HB, Infanrix Hep B, Comvax, DTP-Hib-HBV vaccine, DTP-HBV vaccine, Yi Tai, Heberbiovac HB, Trivac HB, GerVax, DTwP-Hep B-Hib vaccine, Bilive, Hepavax-Gene, SUPERVAX, Comvac5, Shanvac-B, Hebsulin, Recombivax HB, Revac B mcf, Revac B+, Fendrix, DTwP-HepB-Hib, DNA-001, Shan5, Shan6, rhHBsAG vaccine, HBI pentavalent vaccine, LBVD, Infanrix HeXa, YS-HBV-001, IR-101H, TVAX-008, SVF-001, SL-V30, VXX-001 and DtaP-rHB-Hib vaccine.

Examples of HBV therapeutic vaccines include, but are not limited to, HbsAG-HBIG complex, ARB-1598, Bio-Hep-B, NASVAC, abi-HB (intravenous), ABX-203, CP-BNPs, Tetrabhay, GX-110E, GS-4774, peptide vaccine (epsilonPA-44), Hepatrol-07, NASVAC (NASTERAP), IMP-321, BEVAC, Revac B mcf, Revac B+, MGN-1333, KW-2, CVI-HBV-002, AltraHepB, VGX-6200, FP-02, FP-02.2 (HepTeell), NU-500, HBVax, im/TriGrid/antigen vaccine, Mega-CD40L-adjuvanted vaccine, HepB-v, RG7944 (INO-1800), recombinant VLP-based therapeutic vaccine (HBV infection, VLP Biotech), hepatitis B therapeutic DNA vaccine, AdTG-17909, AdTG-17910 AdTG-18202, ChronVac-B, TG-1050, VVX-001, GSK-3528869A (ChAd155-hli-HBV+MVA-HBV+Hbc-HBs/ASO1B-4), VBI-2601, VTP-300 (ChAdOxl-Sii-HBV-Cpmut-TPA-Ssh prime and MVA-Sii-HBV-Cpmut-TPA-Ssh boost), MVA-BN, AVA-2100, HBV-ADV311, YS-HBV-002, TherVacB, JNJ-0535, Lm HBV, GS-2829/GS-6779, GS-6779/HB-400, JNJ-64300535, ISA-204, PRGN-2013, CLB-3000, ASD-253, Hepadvax, GIGA-2339, HEPA-44 and ChAd155-hIi-HBV vaccine. HBV Arenavirus vaccines are described, e.g., in WO2017076988 and WO2017198726.

Examples of telomerase vaccines include, but are not limited to, tertomotide (GV-1001).

### Toll-Like Receptor (TLR) Modulators

In some embodiments, the agent(s) as described herein are combined with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793), TLR11, TLR12 and TLR13.

Examples of TLR modulators include, but are not limited to, AK-0701.

Examples of TLR3 modulators include, but are not limited to, rintatolimod, poly-ICLC, RIBOXXON®, Apoxxim, RIBOXXIM®, IPH-33, MCT-465, MCT-475 and ND-1.1.

Examples of TLR4 agonists include, but are not limited to, G-100, and GSK-1795091.

Example TLR7 agonists that can be co-administered include without limitation RO-7020531, AL-034 (JNJ-4964), DSP-0509, GS-9620 (vesatolimod), LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, ruzotolimod (RG-7854), RG-7795, APR-003, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics).

An TLR7/TLR8 agonist that can be co-administered is NKTR-262, telratolimod and BDB-001.

Examples of TLR-8 inhibitors include, but are not limited to, ZG-170607, ZG-0895.

Example TLR8 agonists that can be co-administered include without limitation E-6887, IMO-4200, IMO-8400, IMO-9200, MCT-465, MEDI-9197, motolimod, resiquimod, GSK-5251738, selgantolimod (GS-9688), CB-06, HRS-9950, SBT-8230, VTX-1463, VTX-763, 3M-051, 3M-052, and the compounds disclosed in US2016289229 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), US20130251673 (Novira Therapeutics), U.S. Pat. No. 9,670,205 (Gilead Sciences, Inc.), US20160289229 (Gilead Sciences, Inc.), WO2017/048727 (Gilead Sciences, Inc.), US20180065938 (Gilead Sciences, Inc.), and US20180086755 (Gilead Sciences, Inc.).

Example TLR9 agonists that can be co-administered include without limitation AST-008, cobitolimod, vidutolimod (CMP-001), IMO-2055, IMO-2125, S-540956, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042.

Examples of TLR7, TLR8 and TLR9 modulators include, but are not limited to, the compounds disclosed in WO2017047769 (Teika Seiyaku), WO2015014815 (Janssen), WO2018045150 (Gilead Sciences Inc), WO2018045144 (Gilead Sciences Inc), WO2015162075 (Roche), WO2017034986 (University of Kansas), WO2018095426 (Jiangsu Hengrui Medicine Co Ltd), WO2016091698 (Roche), WO2016075661 (GlaxoSmithKline Biologicals), WO2016180743 (Roche), WO2018089695 (Dynavax Technologies), WO2016055553 (Roche), WO2015168279 (Novartis), WO2016107536 (Medshine Discovery), WO2018086593 (Livo (Shanghai) Pharmaceutical), WO2017106607 (Merck), WO2017061532 (Sumitomo Dainippon Pharma), WO2016023511 (Chia Tai Tianqing Pharmaceutical), WO2017076346 (Chia Tai Tianqing Pharmaceutical), WO2017046112 (Roche), WO2018078149 (Roche), WO2017040233 (3M Co), WO2016141092 (Gilead Sciences), WO2018049089 (BristolMyers Squibb), WO2015057655 (Eisai Co Ltd), WO2017001307 (Roche), WO2018005586 (BristolMyers Squibb), WO201704023 (3M Co), WO2017163264 (Council of Scientific and Industrial Research (India)), WO2018046460 (GlaxoSmithKline Biologicals), WO2018047081 (Novartis), WO2016142250 (Roche), WO2015168269 (Novartis), WO201804163 (Roche), WO2018038877 (3M Co), WO2015057659 (Eisai Co Ltd), WO2017202704 (Roche), WO2018026620 (BristolMyers Squibb), WO2016029077 (Janus Biotherapeutics), WO201803143 (Merck), WO2016096778 (Roche), WO2017190669 (Shanghai De Novo Pharmatech), U.S. Ser. No. 09/884,866 (University of Minnesota), WO2017219931 (Sichuan KelunBiotech Biopharmaceutical), WO2018002319 (Janssen Sciences), WO2017216054 (Roche), WO2017202703 (Roche), WO2017184735 (IFM Therapeutics), WO2017184746 (IFM Therapeutics), WO2015088045 (Takeda Pharmaceutical), WO2017038909 (Takeda Pharmaceutical), WO2015095780 (University of Kansas), and WO2015023958 (University of Kansas).

In some embodiments, an agent(s) as described herein is co-administered with a TLR7, TLR8 or TLR9 agonist.

### Interferon Alpha Receptor Ligands

Examples of interferon alpha receptor ligands include interferon alpha-2b (INTRON A®), pegylated interferon alpha-2a (PEGASYS®), PEGylated interferon alpha-1b, interferon alpha 1b (HAPGEN®), Veldona, Infradure, Roferon-A, YPEG-interferon alfa-2a (YPEG-rhIFNalpha-2a), P-1101, Algeron, Alfarona, Ingaron (interferon gamma), rSIFN-co (recombinant super compound interferon), Ypeginterferon alfa-2b (YPEG-rhIFNalpha-2b), MOR-22, peginterferon alfa-2b (PEG-INTRON®), Bioferon, Novaferon, Inmutag (Inferon), MULTIFERON®, interferon alfa-n1 (HUMOFERON®), interferon beta-1a (AVONEX®), Shaferon, interferon alfa-2b (Axxo), Alfaferone, interferon alfa-2b (BioGeneric Pharma), interferon-alpha 2 (CJ), Laferonum, VIPEG, BLAUFERON-A, BLAUFERON-B, Intermax Alpha, Realdiron, Lanstion, Pegaferon, Pdferon-B, interferon alfa-2b (IFN, Laboratorios Bioprofarma), alfainterferona 2b, Kalferon, Pegnano, Feronsure, PegiHep, interferon alfa 2b (Zydus-Cadila), interferon alfa 2a, Optipeg A, Realfa 2B, Reliferon, interferon alfa-2b (Amega), interferon alfa-2b (Virchow), ropeginterferon alfa-2b, rHSA-IFN alpha-2a (recombinant human serum albumin intereferon alpha 2a fusion protein), PEG-IFN-alpha, rHSA-IFN alpha 2b, recombinant human interferon alpha-(1b, 2a, 2b), peginterferon alfa-2b (Amega), peginterferon alfa-2a, Reaferon-EC, Proquiferon, Uniferon, Urifron, interferon alfa-2b (Changchun Institute of Biological Products), Anterferon, Shanferon, Layfferon, Shang Sheng Lei Tai, INTEFEN, SINOGEN, Fukangtai, Pegstat, rHSA-IFN alpha-2b, SFR-9216, BMS-914143, peginterferon alfacon-2, IFNAR agonist, PSP-0012, and Interapo (Interapa).

### CD3 Modulators

Examples of CD3 modulators include, but are not limited to, IMC-1109V, IMC-M113V, anti-HBVxCD3.

### Diacylglycerol Kinase Alpha Inhibitors

Examples of diacylglycerol kinase alpha (DGKa) inhibitors include, but are not limited to, GS-9911, ASP-1570, BAY-2965501.

### Cyclophilin Inhibitors

Examples of cyclophilin inhibitors include, but are not limited to, rencofilstat (CPI-431-32), EDP-494, OCB-030, SCY-635, NVP-015, NVP-018, NVP-019, STG-175, and the compounds disclosed in U.S. Pat. No. 8,513,184 (Gilead Sciences), US20140030221 (Gilead Sciences), US20130344030 (Gilead Sciences), and US20130344029 (Gilead Sciences).

### Thymosin Agonists

Examples of thymosin agonists include, but are not limited to, Thymalfasin, and recombinant thymosin alpha 1 (GeneScience).

### Interleukin Agonists

In some embodiments, the agents described herein are combined with an interleukin agonist, such as IL-2, IL-7, IL-15, IL-10, IL-12 agonists; examples of IL-2 agonists such as proleukin (aldesleukin, IL-2); pegylated IL-2 (eg NKTR-214); modified variants of IL-2 (eg THOR-707), bempegaldesleukin, AIC-284, ALKS-4230, CUI-101, Neo-2/15, AB-359; examples of IL-15 agonists, such as ALT-803, NKTR-255, and hetIL-15, interleukin-15/Fc fusion protein, AM-0015, NIZ-985, SO-C101, IL-15 Synthorin (pegylated 11-15), P-22339, and a IL-15-PD-1 fusion protein N-809; examples of IL-7 include CYT-107.STING agonists, RIG-I and NOD2 modulators.

In some embodiments, the agents described herein are combined with a stimulator of interferon genes (STING). In some embodiments, the STING receptor agonist or activator is selected from the group consisting of ADU-S100 (MIW-815), SB-11285, MK-1454, SR-8291, AdVCA0848, STINGVAX, GSK-532, SYN-STING, MSA-1, SR-8291, 5,6-dimethylxanthenone-4-acetic acid (DMXAA), cyclic-GAMP (cGAMP) and cyclic-di-AMP. In some embodiments, the agents described herein are combined with a RIG-I modulator such as RGT-100, or NOD2 modulator, such as SB-9200, and IR-103.

Examples of STING agonists include, but are not limited to, the compounds disclosed in WO 2018065360 (“Biolog Life Science Institute Forschungslabor und Biochemica-Vertrieb GmbH, Germany), WO 2018009466 (Aduro Biotech), WO 2017186711 (InvivoGen), WO 2017161349 (Immune Sensor), WO 2017106740 (Aduro Biotech), US 20170158724 (Glaxo Smithkiline), WO 2017075477 (Aduro Biotech), US 20170044206 (Merck), WO 2014179760 (University of California), WO2018098203 (JanssnJanssen), WO2018118665 (Merck), WO2018118664 (Merck), WO2018100558 (Takeda), WO2018067423 (Merck), and WO2018060323 (Boehringer).

### Retinoic Acid-Inducible Gene 1 Stimulators

Examples of stimulators of retinoic acid-inducible gene 1 include, but are not limited to, inarigivir soproxil (SB-9200), SB-40, SB-44, ORI-7246, ORI-9350, ORI-7537, ORI-9020, ORI-9198, ORI-7170, and RGT-100.

### NOD2 Stimulators

Examples of stimulators of NOD2 include, but are not limited to, inarigivir soproxil (SB-9200).

iii. Immune Checkpoint Modulators

In some embodiments, the agent(s) as described herein, are combined with one or more blockers or inhibitors of inhibitory immune checkpoint proteins or receptors and/or with one or more stimulators, activators, or agonists of one or more stimulatory immune checkpoint proteins or receptors. Blockade or inhibition of inhibitory immune checkpoints can positively regulate T-cell or NK cell activation and prevent immune escape of infected cells. Activation or stimulation of stimulatory immune check points can augment the effect of immune checkpoint inhibitors in infective therapeutics. In various embodiments, the immune checkpoint proteins or receptors regulate T cell responses (e.g., reviewed in Xu et al., J Exp C/in Cancer Res. (2018) 37:110). In various embodiments, the immune checkpoint proteins or receptors regulate NK cell responses (e.g., reviewed in Davis et al., Semin Immunol. (2017) 31:64-75 and Chiossone et al., Nat Rev Immunol. (2018) 18(11):671-688).

Examples of immune checkpoint proteins or receptors include without limitation CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD 112); CD226 (DNAM-1); Poliovirus receptor (PVR) cell adhesion molecule (PVR, CD155); PVR related immunoglobulin domain containing (PVRIG, CDI12R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBP1); UL16 binding protein 2 (ULBP2); UL16 binding protein 3 (ULBP3); retinoic acid early transcript 1E (RAET1E; ULBP4); retinoic acid early transcript 1G (RAET1G; ULBP5); retinoic acid early transcript 1L (RAET1L; ULBP6); lymphocyte activating 3 (CD223); killer cell immunoglobulin like receptor, three Ig domains and long cytoplasmic tail 1 (KIR, CD158E1); killer cell lectin like receptor C1 (KLRC1, NKG2A, CD159A); killer cell lectin like receptor KI (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); and SLAM family member 7 (SLAMF7).

In some embodiments, the agent(s) described herein are combined with one or more blockers or inhibitors of one or more T-cell inhibitory immune checkpoint proteins or receptors. Illustrative T-cell inhibitory immune checkpoint proteins or receptors include without limitation CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In various embodiments, the agents, as described herein, are combined with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); CD244 (2B4, SLAMF4), Poliovirus receptor (PVR) cell adhesion molecule (PVR, CD155). See, e.g., Xu et al., J Exp C/in Cancer Res. (2018) 37:110.

In some embodiments, the agent(s) as described herein, are combined with one or more blockers or inhibitors of one or more NK-cell inhibitory immune checkpoint proteins or receptors. Illustrative NK-cell inhibitory immune checkpoint proteins or receptors include without limitation killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A); and killer cell lectin like receptor D1 (KLRD1, CD94). In various embodiments, the agents as described herein, are combined with one or more agonist or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor KI (KLRK1, NKG2D, CD314); SLAM family member 7 (SLAMF7). See, e.g., Davis et al., Semin Immunol. (2017) 31:64-75; Fang et al., Semin Immunol. (2017) 31:37-54; and Chiossone et al., Nat Rev Immunol. (2018) 18(11):671-688.

In some embodiments, the one or more immune checkpoint inhibitors comprises a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4. In some embodiments, the small molecule inhibitor of CD274 or PDCD1 is selected from the group consisting of evixapodlin (GS-4224), GS-4416, INCB086550 and MAX10181. Additional examples of small molecule PD-L1 inhibitors include, but are not limited to, those disclosed in U.S. Publication No. US2018305315 (Gilead Sciences), US2020017471 (Gilead Sciences) and US2019270727 (Gilead Sciences). In some embodiments, the small molecule inhibitor of CTLA4 comprises BPI-002.

Examples of inhibitors of CTLA4 that can be co-administered include without limitation ipilimumab, tremelimumab, belatacept, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, AGEN-1884, JHL-1155, KN-044, CG-0161, ATOR-1144, PBI-5D3H5, BPI-002, PSI-001, PRS-010 as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors of PD-L1 (CD274) or PD-1 (PDCD1) that can be co-administered include without limitation pembrolizumab, nivolumab, cemiplimab, pidilizumab, AMP-224, MEDI0680 (AMP-514), spartalizumab, atezolizumab, avelumab, durvalumab, AB-101, ALN-PDL, BMS-936559, CK-301, PF-06801591, BGB-108, BGB-A317 (tislelizumab), GLS-010 (WBP-3055), AK-103 (HX-008), GB-226, AK-105, CS-1003, serplulimab (HLX-10), MGA-012, BI-754091, PDR-001, AGEN-2034, JS-001 (toripalimab), Cetrelimab (JNJ-63723283), genolimzumab (CBT-501), KW-019, LZM-009, BCD-100, LY-3300054, SHR-1201, SHR-1210 (camrelizumab), Sym-021, ABBV-181, PD1-PIK, BAT-1306, RO-7191863 (RO-6084, PD-L1 antisense oligonucleotide), ALG-072571 (PD-L1 targeted siRNA), STI-1110, GX-P2, RG-7446, mDX-400, (MSB0010718C), CX-072, CBT-502, TSR-042 (dostarlimab), MSB-2311, JTX-4014, BGB-A333, SHR-1316, CS-1001 (WBP-3155), MEDI-0680, envafolimab (KN-035), KD-033, KY-1003, IBI-308 (sintilimab), HLX-20, KL-A167, STI-A1014, STI-A1015 (IMC-001), BCD-135, FAZ-053, TQB-2450, MDX1105-01, MSB-0010718C, evixapodlin (GS-4224), GS-4416, INCB086550, MAX10181, ALG-093453, ALG-093702, as well as multi-specific inhibitors FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), GNS-1480 (Epidermal growth factor receptor antagonist; Programmed cell death ligand 1 inhibitor), M-7824 (PD-L1/TGF-β bifunctional fusion protein), REMD-456B (Interferon/PD-L1) and INBRX-105 (4-1BB/PDL1).

Examples of PD-1 inhibitors include, but are not limited to, the compounds disclosed in WO2017112730 (Incyte Corp), WO2017087777 (Incyte Corp), WO2017017624, WO2014151634 (BristolMyers Squibb Co), WO201317322 (BristolMyers Squibb Co), WO2018119286 (Incyte Corp), WO2018119266 (Incyte Corp), WO2018119263 (Incyte Corp), WO2018119236 (Incyte Corp), WO2018119221 (Incyte Corp), WO2018118848 (BristolMyers Squibb Co), WO20161266460 (BristolMyers Squibb Co), WO2017087678 (BristolMyers Squibb Co), WO2016149351 (BristolMyers Squibb Co), WO2015033299 (Aurigene Discovery Technologies Ltd), WO2015179615 (Eisai Co Ltd; Eisai Research Institute), WO2017066227 (BristolMyers Squibb Co), WO2016142886 (Aurigene Discovery Technologies Ltd), WO2016142852 (Aurigene Discovery Technologies Ltd), WO2016142835 (Aurigene Discovery Technologies Ltd; Individual), WO2016142833 (Aurigene Discovery Technologies Ltd), WO2018085750 (BristolMyers Squibb Co), WO2015033303 (Aurigene Discovery Technologies Ltd), WO2017205464 (Incyte Corp), WO2016019232 (3M Co; Individual; Texas A&M University System), WO2015160641 (BristolMyers Squibb Co), WO2017079669 (Incyte Corp), WO2015033301 (Aurigene Discovery Technologies Ltd), WO2015034820 (BristolMyers Squibb Co), WO2018073754 (Aurigene Discovery Technologies Ltd), WO2016077518 (BristolMyers Squibb Co), WO2016057624 (BristolMyers Squibb Co), WO2018044783 (Incyte Corp), WO2016100608 (BristolMyers Squibb Co), WO2016100285 (BristolMyers Squibb Co), WO2016039749 (BristolMyers Squibb Co), WO2015019284 (Cambridge Enterprise Ltd), WO2016142894 (Aurigene Discovery Technologies Ltd), WO2015134605 (BristolMyers Squibb Co), WO2018051255 (Aurigene Discovery Technologies Ltd), WO2018051254 (Aurigene Discovery Technologies Ltd), WO2017222976 (Incyte Corp), WO2017070089 (Incyte Corp), WO2018044963 (BristolMyers Squibb Co), WO2013144704 (Aurigene Discovery Technologies Ltd), WO2018013789 (Incyte Corp), WO2017176608 (BristolMyers Squibb Co), WO2018009505 (BristolMyers Squibb Co), WO2011161699 (Aurigene Discovery Technologies Ltd), WO2015119944 (Incyte Corp; Merck Sharp & Dohme Corp), WO2017192961 (Incyte Corp), WO2017106634 (Incyte Corp), WO2013132317 (Aurigene Discovery Technologies Ltd), WO2012168944 (Aurigene Discovery Technologies Ltd), WO2015036927 (Aurigene Discovery Technologies Ltd), WO2015044900 (Aurigene Discovery Technologies Ltd), and WO2018026971 (Arising International).

In various embodiments, the agents as described herein are combined with anti-TIGIT antibodies, such as BMS-986207, RG-6058, and AGEN-1307.

iv. TNF Receptor Superfamily (TNFRSF) Member Agonists or Activators

In various embodiments, the agents as described herein are combined with an agonist of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Example anti-TNFRSF4 (OX40) antibodies that can be co-administered include without limitation, MEDI6469, MEDI6383, MEDI0562 (tavolixizumab), MOXR0916, PF-04518600, RG-7888, GSK-3174998, INCAGN1949, BMS-986178, GBR-8383, ABBV-368, IBI-101 and those described in WO2016179517, WO2017096179, WO2017096182, WO2017096281, and WO2018089628.

Example anti-TNFRSF5 (CD40) antibodies that can be co-administered include without limitation RG7876, SEA-CD40, APX-005M and ABBV-428.

In some embodiments, the anti-TNFRSF7 (CD27) antibody varlilumab (CDX-1127) is co-administered.

Example anti-TNFRSF9 (4-1BB, CD137) antibodies that can be co-administered include without limitation urelumab, utomilumab (PF-05082566), AGEN2373 and ADG-106.

Example anti-TNFRSF18 (GITR) antibodies that can be co-administered include without limitation, MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628. LAG-3 and TIM-3 inhibitors.

In some embodiments, the agent(s) as described herein are combined with an anti-TIM-3 antibody, such as TSR-022, LY-3321367, MBG-453, and INCAGN-2390.

In some embodiments, the agent(s) described herein are combined with an anti-LAG-3 (Lymphocyte-activation) antibody, such as relatlimab (ONO-4482), LAG-525, MK-4280, REGN-3767, and INCAGN2385.

v. Inhibitor of Apoptosis Proteins Family Proteins (APs)

Examples of IAP inhibitors include, but are not limited to, APG-1387.

vi. Bi- and Tri-Specific Natural Killer (NK)-Cell Engagers

In some embodiments, the agents as described herein, are combined with a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16A, C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FcγR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (41BB). As appropriate, the anti-CD16 binding bi-specific molecules may or may not have an Fc. Illustrative bi-specific NK-cell engagers that can be co-administered target CD16 and one or more HBV-associated antigens as described herein. BiKEs and TriKEs are described, e.g., in Felices, et al., Methods Mol Biol. (2016) 1441:333-346; Fang, et al., Semin Immunol. (2017) 31:37-54. This example is for once, but it explains the platform.

vii. Long Acting Treatments

Long acting entecavir (subcutaneous depot), long acting tenofovir (TFD and TAF) implants (devices) or subcutaneous depot. An example of long acting entecavir is described in Exploration of long-acting implant formulations of hepatitis B drug entecavir, Eur J Pharm Sci. 2019 Aug. 1; 136:104958.

viii. Gene Therapy and Cell Therapy

In some embodiments, the agents described herein are combined with a gene or cell therapy regimen. Gene therapy and cell therapy include without limitation the genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; genetic approaches to modify cellular activity to further alter endogenous immune responsiveness against the infection.

ix. Gene Editors

The genome editing system is selected from the group consisting of: a CRISPR/Cas9 system, a zinc finger nuclease system, a TALEN system, a homing endonucleases system, and a meganuclease system (e.g., an ARCUS system); e.g., cccDNA elimination via targeted cleavage, and altering one or more of the hepatitis B virus (HBV) viral genes. Altering (e.g., knocking out and/or knocking down) the PreC, C, X PreSI, PreS2, S, P or SP gene refers to (1) reducing or eliminating PreC, C, X PreSI, PreS2, S, P or SP gene expression, (2) interfering with Precore, Core, X protein, Long surface protein, middle surface protein, S protein (also known as HBs antigen and HBsAg), polymerase protein, and/or Hepatitis B spliced protein function (HBe, HBc, HBx, PreS1, PreS2, S, Pol, and/or HBSP or (3) reducing or eliminating the intracellular, serum and/or intraparenchymal levels of HBe, HBc, HBx, LHBs, MHBs, SHBs, Pol, and/or HBSP proteins. Knockdown of one or more of the PreC, C, X PreSI, PreS2, S, P and/or SP gene(s) is performed by targeting the gene(s) within HBV cccDNA and/or integrated HBV DNA. Additional examples genome editing systems include, but are not limited to, those disclosed in US2019284543 (Gilead Sciences), and US2019338263 (Gilead Sciences).

Examples of gene therapy, includes, but is not limited to, PBGENE-HBV, or using CRISPR/Cas9 gene editing technology, TG-HBV, EBT-107, CRISPR-Cas12 gene therapy, or EBT-106 (LNP-delivered CRISPR/CasX nuclease).

x. CAR-T Cell Therapy

CAR-T cell therapy includes, but is not limited to, a population of immune effector cells engineered to express a chimeric antigen receptor (CAR), wherein the CAR includes an HBV antigen-binding domain. In certain embodiments, the antigen-binding domain is a domain disclosed herein. In certain embodiments, the antigen-binding domain is other than a domain disclosed herein. In certain embodiments, the antigen is HBsAg (i.e., HbsAg-CART). The immune effector cell is a T-cell or an NK cell. In certain embodiments, the T-cell is a CD4+ T-cell, a CD8+ T-cell, a NK cell or a combination thereof. Cells can be autologous or allogeneic. An example of a CART directed to HBV is described in Cytotherapy. 2018 May; 20(5):697-705. doi: 10.1016/j.jcyt.2018.02.

xi. TCR-T Cell Therapy

TCR-T cell therapy includes, but is not limited to, T cells expressing HBV-specific T cell receptors. TCR-T cells are engineered to target HBV derived peptides presented on the surface of virus-infected cells. An example of a TCR directed to HBV is described in Wisskirchen, K. et al T cell receptor grafting allows virological control of hepatitis B virus infection. J Clin Invest. 2019; 129(7):2932-2945.

TCR-T cell therapy includes, but is not limited to, T-Cells expressing HBV surface antigen (HBsAg)-specific TCR, such as YT-HBV-x, Liocyx-M.

TCR-T cell therapy includes, but is not limited to, TCR-T therapy directed to treatment of HBV, such as LTCR-H2-1 (LT-C21), ALVR-107, SQZ-APC-HBV.

In some embodiments, an agent(s) disclosed herein, or a pharmaceutically acceptable salt thereof, is combined with an HBV DNA polymerase inhibitor, one or two additional therapeutic agents selected from the group consisting of immunomodulators, TLR modulators, HBsAg inhibitors, HBsAg secretion or assembly inhibitors, HBV therapeutic vaccines, HBV antibodies including HBV antibodies targeting the surface antigens of the hepatitis B virus and bispecific antibodies and “antibody-like” therapeutic proteins (such as DARTs®, DUOBODIES®, BITES®, XmAbs®, TandAbs®, Fab derivatives, or TCR-like antibodies), cyclophilin inhibitors, stimulators of retinoic acid-inducible gene 1, stimulators of RIG-I like receptors, PD-1 inhibitors, PD-L1 inhibitors, Arginase inhibitors, PI3K inhibitors, IDO inhibitors, and stimulators of NOD2, and one or two additional therapeutic agents selected from the group consisting of HBV viral entry inhibitors, NTCP inhibitors, HBx inhibitors, cccDNA inhibitors, HBV antibodies targeting the surface antigens of the hepatitis B virus, siRNA, miRNA gene therapy agents, sshRNAs, KDM5 inhibitors, and nucleoprotein modulators (HBV core or capsid protein modulators).

In some embodiments, an agent(s) disclosed herein, or a pharmaceutically acceptable salt thereof, is combined with at least a second additional therapeutic agent selected from the group consisting of: HBV DNA polymerase inhibitors, immunomodulator, TLR modulators, HBsAg inhibitors, HBV therapeutic vaccines, HBV antibodies including HBV antibodies targeting the surface antigens of the hepatitis B virus and bispecific antibodies and “antibody-like” therapeutic proteins (such as DARPins®, anti-pMHC TCR-like antibodies, DARTs®, DUOBODIES®, BITES®, XmAbs®, TandAbs®, Fab derivatives, or TCR-like antibodies), cyclophilin inhibitors, stimulators of retinoic acid-inducible gene 1, stimulators of RIG-I like receptors, PD-1 inhibitors, PD-L1 inhibitors, Arginase inhibitors, PI3K inhibitors, IDO inhibitors, and stimulators of NOD2.

In some embodiments, an agent(s) disclosed herein, or a pharmaceutically acceptable salt thereof, is combined with at least a second additional therapeutic agent selected from the group consisting of: HBV DNA polymerase inhibitors, HBV viral entry inhibitors, NTCP inhibitors, HBx inhibitors, cccDNA inhibitors, HBV antibodies targeting the surface antigens of the hepatitis B virus, siRNA, miRNA gene therapy agents, sshRNAs, KDM5 inhibitors, and nucleoprotein modulators (HBV core or capsid protein inhibitors).

In some embodiments, an agent(s) disclosed herein, or a pharmaceutically acceptable salt thereof, is combined with compounds such as those disclosed in U.S. Publication No. 2010/0143301 (Gilead Sciences), U.S. Publication No. 2011/0098248 (Gilead Sciences), U.S. Publication No. 2009/0047249 (Gilead Sciences), U.S. Pat. No. 8,722,054 (Gilead Sciences), U.S. Publication No. 2014/0045849 (Janssen), U.S. Publication No. 2014/0073642 (Janssen), WO2014/056953 (Janssen), WO2014/076221 (Janssen), WO2014/128189 (Janssen), U.S. Publication No. 2014/0350031 (Janssen), WO2014/023813 (Janssen), U.S. Publication No. 2008/0234251 (Array Biopharma), U.S. Publication No. 2008/0306050 (Array Biopharma), U.S. Publication No. 2010/0029585 (Ventirx Pharma), U.S. Publication No. 2011/0092485 (Ventirx Pharma), US2011/0118235 (Ventirx Pharma), U.S. Publication No. 2012/0082658 (Ventirx Pharma), U.S. Publication No. 2012/0219615 (Ventirx Pharma), U.S. Publication No. 2014/0066432 (Ventirx Pharma), U.S. Publication No. 2014/0088085 (Ventirx Pharma), U.S. Publication No. 2014/0275167 (Novira Therapeutics), U.S. Publication No. 2013/0251673 (Novira Therapeutics), U.S. Pat. No. 8,513,184 (Gilead Sciences), U.S. Publication No. 2014/0030221 (Gilead Sciences), U.S. Publication No. 2013/0344030 (Gilead Sciences), U.S. Publication No. 2013/0344029 (Gilead Sciences), US20140275167 (Novira Therapeutics), US20130251673 (Novira Therapeutics), U.S. Publication No. 2014/0343032 (Roche), WO2014037480 (Roche), U.S. Publication No. 2013/0267517 (Roche), WO2014131847 (Janssen), WO2014033176 (Janssen), WO2014033170 (Janssen), WO2014033167 (Janssen), WO2015/059212 (Janssen), WO2015118057 (Janssen), WO2015011281 (Janssen), WO2014184365 (Janssen), WO2014184350 (Janssen), WO2014161888 (Janssen), WO2013096744 (Novira), US20150225355 (Novira), US20140178337 (Novira), US20150315159 (Novira), US20150197533 (Novira), US20150274652 (Novira), US20150259324, (Novira), US20150132258 (Novira), U.S. Pat. No. 9,181,288 (Novira), WO2014184350 (Janssen), WO2013144129 (Roche), US20100015178 (Incyte), US2016137652 (Flexus Biosciences, Inc.), WO2014073738 (Flexus Biosciences, Inc.), WO2015188085 (Flexus Biosciences, Inc.), U.S. Publication No. 2014/0330015 (Ono Pharmaceutical), U.S. Publication No. 2013/0079327 (Ono Pharmaceutical), U.S. Publication No. 2013/0217880 (Ono pharmaceutical), WO2016057924 (Genentech/Constellation Pharmaceuticals), US20140275092 (Genentech/Constellation Pharmaceuticals), US20140371195 (Epitherapeutics) and US20140371214 (Epitherapeutics), US20160102096 (Epitherapeutics), US20140194469 (Quanticel), US20140171432, US20140213591 (Quanticel), US20160039808 (Quanticel), US20140275084 (Quanticel), WO2014164708 (Quanticel), U.S. Pat. No. 9,186,337B2 (Oryzon Genomics), and other drugs for treating HBV, and combinations thereof.

In some embodiments, an agent(s) disclosed herein, or a pharmaceutically acceptable salt thereof, is combined with 5-30 mg tenofovir alafenamide fumarate, tenofovir alafenamide hemifumarate, or tenofovir alafenamide. In some embodiments, an agent disclosed herein, or a pharmaceutically acceptable salt thereof, is combined with 5-10; 5-15; 5-20; 5-25; 25-30; 20-30; 15-30; or 10-30 mg tenofovir alafenamide fumarate, tenofovir alafenamide hemifumarate, or tenofovir alafenamide. In some embodiments, an agent disclosed herein, or a pharmaceutically acceptable salt thereof, is combined with 10 mg tenofovir alafenamide fumarate, tenofovir alafenamide hemifumarate, or tenofovir alafenamide. In some embodiments, an agent disclosed herein, or a pharmaceutically acceptable salt thereof, is combined with 25 mg tenofovir alafenamide fumarate, tenofovir alafenamide hemifumarate, or tenofovir alafenamide. An agent(s) as disclosed herein may be combined with the agents provided herein in any dosage amount of the compound (e.g., from 50 mg to 500 mg of compound) the same as if each combination of dosages were specifically and individually listed.

In some embodiments, an agent(s) disclosed herein, or a pharmaceutically acceptable salt thereof, is combined with 100-400 mg tenofovir disoproxil fumarate, tenofovir disoproxil hemifumarate, or tenofovir disoproxil. In some embodiments, an agent(s) disclosed herein, or a pharmaceutically acceptable salt thereof, is combined with 100-150; 100-200, 100-250; 100-300; 100-350; 150-200; 150-250; 150-300; 150-350; 150-400; 200-250; 200-300; 200-350; 200-400; 250-350; 250-400; 350-400 or 300-400 mg tenofovir disoproxil fumarate, tenofovir disoproxil hemifumarate, or tenofovir disoproxil. In some embodiments, an agent(s) disclosed herein, or a pharmaceutically acceptable salt thereof, is combined with 300 mg tenofovir disoproxil fumarate, tenofovir disoproxil hemifumarate, or tenofovir disoproxil. In some embodiments, an agent(s) disclosed herein, or a pharmaceutically acceptable salt thereof, is combined with 250 mg tenofovir disoproxil fumarate, tenofovir disoproxil hemifumarate, or tenofovir disoproxil. In some embodiments, an agent(s) disclosed herein, or a pharmaceutically acceptable salt thereof, is combined with 150 mg tenofovir disoproxil fumarate, tenofovir disoproxil hemifumarate, or tenofovir disoproxil. An agent(s) as disclosed herein may be combined with the agents provided herein in any dosage amount of the compound (e.g., from 50 mg to 500 mg of compound) the same as if each combination of dosages were specifically and individually listed.

xii. Other HBV Drugs

Examples of other drugs for the treatment of HBV include, but are not limited to, alpha-hydroxytropolones, amdoxovir, antroquinonol, beta-hydroxycytosine nucleosides, ARB-199, CCC-0975, ccc-R08, CKD-388, DF-006, elvucitabine, ezetimibe, cyclosporin A, gentiopicrin (gentiopicroside), HH-003, hepalatide, ISR-51, JNJ-56136379, M-1428, nitazoxanide, birinapant, NJK14047, NOV-205 (molixan, BAM-205), oligotide, mivotilate, feron, GST-HG-131, levamisole, Ka Shu Ning, alloferon, WS-007, Y-101 (Ti Fen Tai), PEG-IIFNm, KW-3, BP-Inter-014, oleanolic acid, HepB-nRNA, cTP-5 (rTP-5), HSK-II-2, HEISCO-106-1, HEISCO-106, Hepbarna, IBPB-0061A, Hepuyinfen, DasKloster 0014-01, ISA-204, Jiangantai (Ganxikang), MIV-210, OB-AI-004, PF-06, picroside, DasKloster-0039, hepulantai, IMB-2613, NCO-48 Fumarate, SFA-001, TCM-800B, TQA-3810, VRON-0200, ZYF-0057, JNJ-7744, HPG-3466, HPG-6189, HPG-4892, GS-4321, GS-6451, HRS-5635, LW-231, PA-3670, ABP-6016, MBT-1316, HNC-280, Ab-MVP, HT-103, SCG-211, BW-20507, DA-2803, VD-1219, AK-0706, HDM-817101, HDM-10828122, HDM-8421034, reduced glutathione, RO-6864018, ENOB-HB-01, RG-7834, QL-007, sofosbuvir, ledipasvir, UB-551, PA-1010, HPN-BV1, STSG-0002, and ZH-2N, and the compounds disclosed in US20150210682, (Roche), US 2016/0122344 (Roche), WO2015173164, WO2016023877, US2015252057A (Roche), WO16128335A1 (Roche), WO16120186A1 (Roche), US2016237090A (Roche), WO16107833A1 (Roche), WO16107832A1 (Roche), US2016176899A (Roche), WO16102438A1 (Roche), WO16012470A1 (Roche), US2016220586A (Roche), and US2015031687A (Roche).

### EXAMPLES

The invention will be described in greater detail by way of specific examples. The following examples are offered for illustrative purposes and are not intended to limit the invention in any manner. Those of skill in the art will readily recognize a variety of non-critical parameters which can be changed or modified to yield essentially the same results.

### Example 1. HDV Antiviral Activity of HBV Targeting siRNA in an HBV/HDV Co-Infected PHH Spread Assay

The anti-HDV activity of an HBV siRNA targeting HBx sequence was evaluated in the HBV/HDV co-infected PHH spread assay. Cryopreserved primary human hepatocytes (PHH) were thawed and cells were recovered by centrifugation at 100×g (Beckman Coulter, Allegra X-14R) through cryopreserved hepatocyte recovery medium (Thermo Fisher Scientific; CM7500) and plated in collagen coated 96-well plates (Thermo Fisher Scientific; CM1096) at a density of 70,000 live cells per well. Cells were plated in William's E medium (Thermo Fisher Scientific; A1217601) supplemented with 3.6% hepatocyte thawing and plating supplement (Thermo Fisher Scientific, A15563), 5% fetal bovine serum (Thermo Fisher Scientific; 16000-036), 1 μM dexamethasone (Thermo Fisher Scientific, A15563), and 0.2% Torpedo antibiotic mix (Bioreclamation IVT; Z990008). Approximately 24 hours after plating, plating medium was removed and cells were refreshed with maintenance medium: William's E medium (Thermo Fisher Scientific; A1217601) supplemented with 4% hepatocyte maintenance supplement (Thermo Fisher Scientific; AI15564), 2% fetal bovine serum (Thermo Fisher Scientific; 16000-036), 0.1 μM dexamethasone (Thermo Fisher Scientific; A115564), 1.5% DMSO (Sigma-Aldrich, St. Louis, MO; D8418), and 0.2% Torpedo antibiotic mix (BioreclamationIVT; Z990008). Production of HBV AD38 (genotype D) virions from HepAD38 cells was performed as previously described (Decorsiere, A., Mueller, H., van Breugel, P. C., Abdul, F., Gerossier, L., Beran, R. K., Livingston, C. M., Niu, C., Fletcher, S. P., Hantz, O., and Strubin, M. Hepatitis B Virus X Protein Identifies the Smc5/6 Complex as a Viral Restriction Factor. Nature 2016).

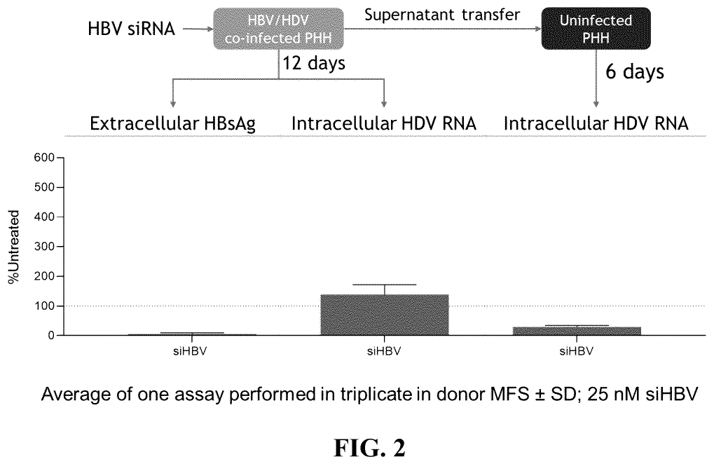

For HBV/HDV co-infection, PHH were infected with HBV GTD-AD38 using 1000 genome equivalents (GE) per cell in 4% PEG medium as described above. Four days later, PHH were infected with HDV at an MOI of 2 in 4% PEG medium. One day post-HDV infection, HBV siRNA (5′ GGAGCAAACAUUAUCGGGA 3′) (SEQ ID NO:7) targeting the HBx sequence was transfected at 25 nM final concentration according to the manufacturer's instructions (Life Technologies). siCtrl is a non-targeting siRNA (5′ GAUCGUAUCUAUCUAUUAU 3′) (SEQ ID NO:8). The medium was collected 7 days post-transfection with siRNAs and extracellular HBsAg was measured by an electrochemiluminescence assay (MSD). The antigen concentration in each sample was calculated by interpolation from a standard curve with purified antigen. The MSD assay was performed according to the manufacturer's instructions (Meso Scale Diagnostics, Rockville, MD). To measure HDV spread, the collected medium was subsequently used to infect naïve PHH on a new 96-well plate in maintenance medium containing 4% PEG final concentration. The medium was replaced with fresh maintenance medium without PEG 24 hours post-infection and again 4 days post-infection. Following 7 days of incubation, the medium was collected and extracellular HBsAg levels were measured by MSD. Total cellular RNA was isolated from both the first and second parts of the co-infected/spread assay using the RNeasy 96 Kit (Qiagen) following the manufacturer's instruction. Quantification of total HDV RNA by qRT-PCR (quantitative reverse transcription polymerase chain reaction) amplification was performed by combining 5 μL of RNA to HDV forward primer (GTAGACTCCGGACCTAGGAAGAG) (SEQ ID NO:9), reverse primer (CTTCCTTCGTCGGTGATCCT) (SEQ ID NO: 10), and probe (CCTTGTCGGTGAATCC, FAM-NFQ) (SEQ ID NO:11), and GAPDH primer and probe set (Thermo catalog #4310884E) and 1× TaqMan Fast Virus 1-Step Master Mix (Thermo Fisher Scientific; 4444434) for a total reaction volume of 20 μL in 96-well PCR plates (Thermo Fisher Scientific; 4346906). qRT-PCR was carried out on a real-time PCR system (Thermo Fisher Scientific; QuantStudio 7 Flex) using the following conditions: 50° C. for 5 minutes, then 95° C. for 20 seconds, followed by 40 cycles of 95° C. for 3 seconds and 60° C. for 30 seconds. GAPDH mRNA expression was used to normalize target gene expression. Levels of HDV RNA were calculated as fold change relative to DMSO treated sample using the 2−ΔΔCt method (Livak K J, Schmittgen T D. Analysis of relative gene expression data using real-time quantitative PCR and the 2(−Delta Delta C(T)) Method. Methods 2001). Treatment with HBV siRNA demonstrated a reduction in HDV spread following supernatant transfer, as shown in FIG. 2.

### Example 2. HDV Antiviral Activity of Bulevirtide (BLV) in Combination with HBV Targeting siRNA in HBV/HDV Co-Infected Primary Human Hepatoctyes (PHH)

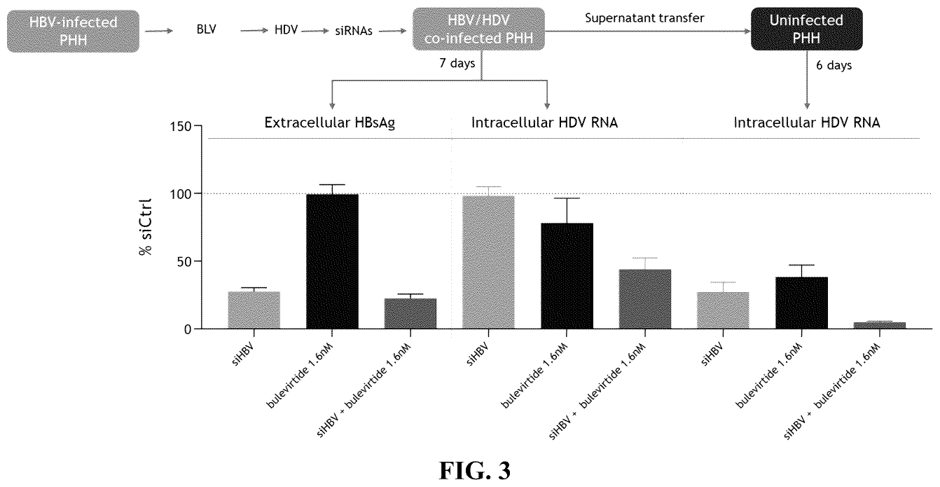

The antiviral activity of BLV in combination with an HBV-targeting siRNA was evaluated in the HBV/HDV co-infected PHH spread assay. Cryopreserved PHH were thawed and cells were recovered as described above. For HBV/HDV co-infection, PHH were infected with HBV GTD-AD38 using 1000 genome equivalents per cell in 4% PEG medium as described above. Four days later, PHH were infected with HDV at an MOI of 2 in 4% PEG medium. In cases where BLV was added, 2×EC50 (1.6 nM) BLV was incubated with HBV-infected PHH for an hour at 37° C. and then the PHH were infected with HDV. One day post-HDV infection, HBV siRNA or a non-targeting control (siCtrl) was transfected at 25 nM final concentration as previously described. Medium was collected 7 days post transfection and extracellular supernatant used to subsequently infected naïve PHH on a new 96-well plate in maintenance medium as described above. Extracellular HBsAg and intracellular HDV RNA assayed as described previously and normalized to the siCtrl. Both HBV siRNA and bulevirtide demonstrated a reduction in HDV spread alone as monitored by intracellular HDV RNA following supernatant transfer. Combination resulted in further reduction in viral spread, as shown in FIG. 3.

### Example 3. HDV Antiviral Activity of Bulevirtide (BLV) in Combination with HBV Targeting siRNA in HBV/HDV Co-Infected Mouse Model

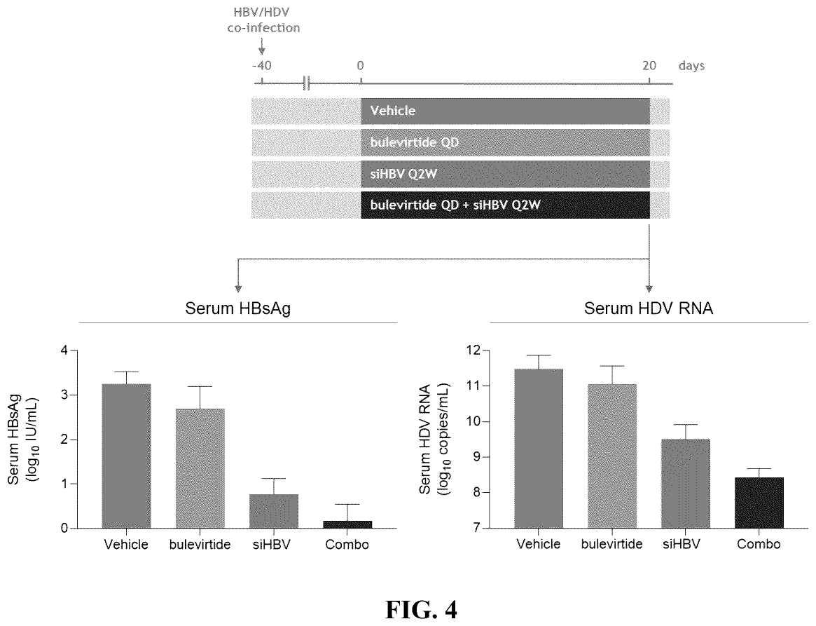

The antiviral activity of BLV in combination with an HBV-targeting siRNA was evaluated in vivo using the HBV/HDV co-infected uPA-SCID mouse model. Human liver-chimeric mice were co-infected with HBV (genotype C, 6×10⁷ copies/mL) and HDV (genotype 2×10×10¹¹ copies/mL) for forty days. Mice were then treated with either 2 mg/kg bulevirtide subcutaneous (QD), 3 mg/kg siHBV (5′ ACCUCUGCCUAAUCAUCUCUU 3′) (SEQ ID NO:12) intravenous (Q2W) or a combination of 2 mg/kg buleviritde subcutaneous (QD) and 3 mg/kg siHBV intravenous (Q2W) for 20 days. Serum HBsAg and HDV RNA was measured at the end of the study as described above. siHBV LNPs were prepared by Precision Nanosystems using the NanoAssemblr Benchtop. Both BLV and HBV siRNA demonstrated a reduction in HBsAg and serum HDV RNA which was further enhanced when combined, as shown in FIG. 4.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A method of treating hepatitis D virus (HDV) infection in a patient, comprising administering to the patient:
i) bulevirtide, or a pharmaceutically acceptable salt thereof; and
ii) an inhibitory nucleic acid targeting hepatitis B virus (HBV).

2. The method of claim 1, wherein the bulevirtide, or a pharmaceutically acceptable salt thereof, is administered subcutaneously.

3. The method of claim 1, wherein the bulevirtide, or a pharmaceutically acceptable salt thereof, is administered subcutaneously in a dosage of about 1 mg to about 5 mg.

4. The method of claim 1, wherein the bulevirtide, or a pharmaceutically acceptable salt thereof, is administered subcutaneously in a dosage of about 2 mg.

5. The method of claim 1, wherein the method comprises administering to the patient:
i) a pharmaceutical composition comprising the bulevirtide, or a pharmaceutically acceptable salt thereof, and one or more pharmaceutically acceptable excipients; and
ii) a pharmaceutical composition comprising an inhibitory nucleic acid targeting hepatitis B virus (HBV), and one or more pharmaceutically acceptable excipients.

6. (canceled)

7. The method of claim 5, wherein the pharmaceutical composition comprising the bulevirtide, or a pharmaceutically acceptable salt thereof, is an injectable solution suitable for subcutaneous administration to the patient.

8. The method of claim 7, wherein the injectable solution comprises about 1 mg to about 5 mg of the bulevirtide, or a pharmaceutically acceptable salt thereof, and one or more pharmaceutically acceptable excipients.

9. The method of claim 7, wherein the injectable solution comprises about 2 mg of the bulevirtide, or a pharmaceutically acceptable salt thereof, and one or more pharmaceutically acceptable excipients.

10. The method of claim 7, wherein the injectable solution comprises about 2 mg on a free base basis of bulevirtide acetate, and one or more pharmaceutically acceptable excipients.

11. The method of claim 7, wherein the injectable solution comprises one or more pharmaceutically acceptable excipients selected from sodium carbonate, sodium bicarbonate, and mannitol; or any combination thereof.

12. (canceled)

13. The method of claim 10, wherein the injectable solution comprises:
about 2 mg on a free base basis of bulevirtide acetate;
about 0.1% w/w to about 0.5% w/w of sodium carbonate;
about 3.5% w/w to about 4.0% w/w of sodium bicarbonate; and
about 90% w/w to about 95% w/w of mannitol.

14. The method of claim 10, wherein the injectable solution comprises:
about 2 mg on a free base basis of bulevirtide acetate;
about 0.25% w/w of sodium carbonate;
about 3.7% w/w of sodium bicarbonate; and
about 92% w/w of mannitol.

15. The method of claim 7, wherein the injectable solution further comprises one or more buffering agents and a solvent, wherein the one or more buffering agents comprise sodium hydroxide and hydrochloric acid and the solvent comprises water.

16-17. (canceled)

18. The method of claim 1, wherein the inhibitory nucleic acid is complementary to at least 8 consecutive nucleic acids of a HBsAg mRNA sequence.

19. The method of claim 1, wherein the inhibitory nucleic acid comprises at least 15 consecutive nucleic acids from the nucleic acid sequence set forth in SEQ ID NO:2, SEQ ID NO:5, or SEQ ID NO:6.

20. The method of claim 1, wherein the inhibitory nucleic acid comprises the nucleic acid sequence ACCUCUGCCUAAUCAUCUC (SEQ ID NO:4).

21. (canceled)

22. The method of claim 1, wherein the inhibitory nucleic acid comprises an antisense oligonucleotide, a short interfering RNA (siRNA), or a short hairpin RNA (shRNA).

23. (canceled)

24. The method of claim 1, wherein the inhibitory nucleic acid comprises one or more modifications.

25-26. (canceled)

27. The method of claim 1, wherein the patient has been identified as having hepatitis B.

28. The method of claim 1, wherein the patient has been identified as having compensated liver disease.


SEQUENCE LISTING (12 sequences)

SEQ ID NO: 1 - protein, 47 aa, synthetic construct
GTNLSVPNPLGFFPDHQLDPAFGANSNNPDWDFNPNKDHWPEANKVG

SEQ ID NO: 2 - DNA, 639 bp, Hepatitis B virus
  source 1..639: mol_type = other DNA
atgcaactttttcacctctgcctaatcatctcatgttcatgtcctactgttcaagcctccaagctgtgccttgggtggctttggggcatggacattgacccgtataaagaatttggagcttctgtggagttactctcttttttgccttctgacttctttccttctattcgagatctcctcgacaccgcctctgctctgtatcgggaggccttagagtctccggaacattgttcacctcaccatacggcactcaggcaagctattttgtgttggggtgagttaatgaatctggccacctgggtgggaagtaatttggaagatccagcatccagggaattagtagtcagttatgtcaacgttaatatgggcctaaaaatcagacaactattgtggtttcacatttcctgtcttacttttgggagagaaactgttcttgaatatttggtgtcttttggagtgtggattcgcactcctcctgcatatagaccaccaaatgcccctatcttatcaacacttccggaaactactgttgttagacgaagaggcaggtcccctagaagaagaactccctcgcctcgcagacgaaggtctcaatcgccgcgtcgcagaagatctcaatctcgggaatctcaatgttag

SEQ ID NO: 3 - RNA, 18 nt, synthetic construct
  source 1..18: mol_type = other RNA
cctctgcctaatcatctc

SEQ ID NO: 4 - RNA, 19 nt, synthetic construct
  source 1..19: mol_type = other RNA
acctctgcctaatcatctc

SEQ ID NO: 5 - DNA, 465 bp, Hepatitis B virus
  source 1..465: mol_type = other DNA
atggctgctaggctgtgctgccaactggatcctgcgcgggacgtcctttgtttacgtcccgtcggcgctgaatcccgcggacgacccctcccggggccgcttggggctctaccgcccgcttctccgcctattgtaccgtccgaccacggggcgcacctctctttacgcggactccccgtctgtgccttctcatctgccggaccgtgtgcacttcgcttcacctctgcacgtcgcatggagaccaccgtgaacgcccacgggaacctgcccaaggtcttgcataagaggactcttggactttcagcaatgtcaacgaccgaccttgaggcatacttcaaagactgtgtgtttactgagtgggaggagttgggggaggaggttaggttaaaggtctttgtactaggaggctgtaggcataaattggtgtgttcaccagcaccatgcaactttttcacctctgcctaa

SEQ ID NO: 6 - DNA, 1202 bp, Hepatitis B virus
  source 1..1202: mol_type = other DNA
atgggaggttggtcatcaaaacctcgcaaaggcatggggacgaatctttctgttcccaaccctctgggattctttcccgatcatcagttggaccctgcattcggagccaactcaaacaatccagattgggacttcaaccccatcaaggaccactggccagcagccaaccaggtaggagtgggagcattcgggccaaggctcacccctccacacggcggtattttgggtggagccctcaggctcagggcatattgaccacagtgtcaacaattcctcctcctgcctccaccaatcggcagtcaggaaggcagcctactcccatctctccacctctaagagacagtcatcctcaggccatgcagtggaattccactgccttccaccaagctctgcaggatcccaaagtcaggggtctgtatcttcctgctggtggctccagttcaggaacagtaaaccctgctccgaatattgcctctcacatctcgtcaatctccgcgaggactggggaccctgtgacgaacatggagaacatcacatcaggattcctaggacccctgctcgtgttacaggcggggtttttcttgttgacaagaatcctcacaataccgcagagtctagactcgtggtggacttctctcaattttctaggggggtcacccgtgtgtcttggccaaaattcgcagtccccaacctccaatcactcaccaacctcctgtcctccaatttgtcctggttatcgctggatgtgtctgcggcgttttatcatattcctcttcatcctgctgctatgcctcatcttcttgttggttcttctggattatcaaggtatgttgcccgtttgtcctctaattccaggatcaacaacaaccagtacgggaccatgcaaaacctgcacgactcctgctcaaggcaactctatgtttccctcatgttgctgtacaaaacctacggatggaaattgcacctgtattcccatcccatcgtcctgggctttcgcaaaatacctatgggagtgggcctcagtccgtttctcttggctcagtttactagtgccatttgttcagtggttcgtagggctttcccccactgtttggctttcagctatatggatgatgtggtattgggggccaagactgtacagcatcgtgagtccctttataccgctgttaccaattttcttttgtctctgggtatacatttaa

SEQ ID NO: 7 - RNA, 19 nt, synthetic construct
  source 1..19: mol_type = other RNA
ggagcaaacattatcggga

SEQ ID NO: 8 - RNA, 19 nt, synthetic construct
  source 1..19: mol_type = other RNA
gatcgtatctatctattat

SEQ ID NO: 9 - DNA, 23 bp, synthetic construct
  source 1..23: mol_type = other DNA
gtagactccggacctaggaagag

SEQ ID NO: 10 - DNA, 20 bp, synthetic construct
  source 1..20: mol_type = other DNA
cttccttcgtcggtgatcct

SEQ ID NO: 11 - DNA, 16 bp, synthetic construct
  source 1..16: mol_type = other DNA
ccttgtcggtgaatcc

SEQ ID NO: 12 - RNA, 21 nt, synthetic construct
  source 1..21: mol_type = other RNA
acctctgcctaatcatctctt